(19) United States
(12) Patent Application Publication
(10) Pub. No.: US 2010/0098706 A1
(43) Pub. Date: Apr. 22, 2010

(54) MONOCLONAL ANTIBODY DIRECTED AGAINST THE HUMAN LDL RECEPTOR

(75) Inventors: Christophe De Romeuf, Lambersart (FR); Christian Behrens, Palaiseau (FR)

Correspondence Address: MARSH, FISCHMANN & BREYFOGLE LLP, 8055 East Tufts Avenue, Suite 450, Denver, CO, 80237, US

(73) Assignee: LFB BIOTECHNOLOGIES, Les Ulis (FR)

(21) Appl. No.: 12/519,275

(22) PCT Filed: Dec. 28, 2007
(86) PCT No.: PCT/FR2007/002187

§ 371 Date: Dec. 11, 2009

(30) Foreign Application Priority Data
Dec. 29, 2006 (FR) 06 11546

(51) Int. Cl. A61K 39/395; C07K 16/00; C07H 21/04; A61P 35/00; C12N 15/63; C12N 5/10

(52) U.S. Cl. 424/144.1; 530/388.22; 530/387.3; 536/23.53; 435/320.1; 435/325; 435/375

(57) ABSTRACT

The invention relates to a monoclonal antibody directed against the human LDL (Low Density Lipoprotein) receptor, in which the variable region of each of the light chains is coded by the murine nucleic acid sequence SEQ ID NO: 5, the variable region of each of the heavy chains is coded by the murine nucleic acid sequence SEQ ID NO: 7, or by nucleic acid sequences having a sufficient homology with the sequences SEQ ID NO: 5 and SEQ ID NO: 7 so that the nature and the affinity of the bond between the antibody and its antigene are not modified, while the constant regions of the light chains and the heavy chains thereof are constant regions from a non-murine species.

Malignant melanoma is a malignant tumour of the pigment system (melanocytes) occurring either originally in healthy skin, or by degeneration of a pre-existing naevus.

Melanoma is the skin cancer which is increasing most in frequency throughout the world. It doubles every 10 years. The majority of cases occur on healthy skin, less than a quarter appear on a pre-existing naevus.

Each year, in the United States, more than 47,000 individuals are newly diagnosed with a melanoma and 7,700 of them die of this aggressive cancer of the skin.

In France, 4,000 to 5,000 cases are discovered every year, and 1,000 individuals die from it.

Early prevention and detection of the melanoma are therefore essential.

In fact, whilst research is being carried out into the treatment of melanoma, the standard treatment of melanoma is still based on surgery. This may involve an initial excision which must comprise variable margins depending on the thickness of the tumour, or surgical repair in order to adapt the excision margins to the thickness of the tumour if necessary. If the check-up reveals one or more metastases, they should be treated either by surgical ablation or by chemotherapy.

Current hopes in the treatment of melanoma are turned towards novel cell therapy approaches.

The rationale for their use is based on two findings: on the one hand, spontaneous regressions of primitive melanomas or of cutaneous metastases have been reported in the literature, leading to the supposition that the immune system plays an important role in the development of this tumour; on the other hand, in the last few years, antigens specific to melanoma tumour cells have been isolated.

The cell therapy used for melanoma involves two types of treatment: adoptive immunotherapy, a process which involves administering medicaments with an “antitumour activity” to a patient in order to stimulate his immune system and help his body to fight the cancer more effectively, by means of “TIL” or (Tumour Infiltrating Lymphocyte), and the active immunotherapy represented by the vaccination.

Adoptive immunotherapy with TIL consists of injecting the patient with a large quantity (several billion) of cytotoxic T cells, specific to the melanoma antigens, which are isolated from a tumour or metastasis and expanded in vitro in a GMP (Good Manufacturing Practice) laboratory. This is therefore an autologous system.

The first clinical studies carried out in the USA with TILs in the metastatic stage of the melanoma obtained a response level of the order of 35%, but often with rapid relapses, leading to discussion of abandoning this passive immunotherapy approach.

However, the concept of effectiveness on a “residual” tumour mass, raised by treatments with cytokines, in particular interferon alpha, has led to the question of its application to the TILs being raised.

In parallel with this TIL approach, the approach of injecting clones directed against melanoma antigens, essentially Melan-A and tyrosinase, is being developed. Preliminary results have been obtained at the metastatic stage, but it has above all been demonstrated that the clones injected into a patient could on the one hand be found at the metastatic site, and on the other hand that they were amplified in patients after injection.

Active immunotherapy by vaccination relates either to the injection of lysates of melanoma cells irradiated subcutaneously (multi-antigen approach), or to the injection of the specific peptides obtained after identification of certain melanoma antigens in an HLA-restricted context. The objective is therefore to induce a specific anti-melanoma immune response.

At present, it is possible schematically to differentiate between 4 generations of vaccines in the melanoma. The first generation is that of the multi-antigen vaccines. They are constituted by the homogenate of irradiated tumour cells. They thus have the advantage of being constituted by several tumour antigens which increases the chances of being able a priori to correspond to one of the cytotoxic T-cell populations presented by the patient.

The melanoma tumour antigens are: either melanocyte differentiation antigens: tyrosinase, gp 100, Melan-A/MART-1, gp 75; or tumour-specific antigens (embryonic antigens): “Melanoma Associated Antigen” MAA: Mage-1, Mage-2, Mage-3, Bage, Gage-1, Gage-2, Muc-1, Rage-1, NA-17. On the other hand, these antigens are generally present in small quantities, which limit their activating action. These tumour homogenates, injected into the patient subcutaneously or intradermally, originate either from the patient himself (autologous system), or from an allogenic tumour line. The stimulating action of the tumour homogenate on the T lymphocyte cells can be intensified by adding an immune adjuvant to the latter. Thus, BCG, Detox, QS-21, and MF-59 have been proposed as adjuvants.

The second generation is that of the specific antigen vaccines, which are based on the principle of the injection of a single tumour antigen into the patient. They therefore induce a significant cytotoxic T lymphocyte activation.

On the other hand, their use is subject to two conditions:

on the one hand, an HLA restriction (HLA-A1 for Mage, HLA-A2 for NA-17, Melan-A); on the other hand, expression by the tumour or metastasis of the antigen corresponding to the peptide that is to be injected.

The lesion must therefore be accessible for a biopsy which makes it possible to identify by PCR the presence or absence of the antigen. This of course limits the possibilities of a patient benefiting from a vaccine. Until now, the clinical protocols have essentially been produced with the peptides Mage-3, Mage-1, Melan-A/Mart-1, NA-17, tyrosinase and NY-SO1.

The third generation of vaccine is the use of dendritic cells for vaccination into the melanoma, and is based on the fact that these cells are excellent antigen-presenting cells. They are capable of internalizing antigens and presenting them to the T lymphocytes in an HLA context of class II for the CD4+ lymphocytes and HLA class I for the cytotoxic lymphocytes. This activation of the T lymphocyte moreover involves co-stimulating molecules CD40-CD40L and B7-CD28. The principle of the development of the treatment is based on the in vitro culture of CD34⁺ cells or of monocytes isolated from the blood of the patient (cytapheresis) in the presence of cytokines such as GM-CSF (Granulocyte-Macrophage Colony-Stimulating Factor and IL-13 (Interleukin-13). The differentiated dendritic cells obtained are then charged in vitro with one or more peptides. These so-called charged dendritic cells then become excellent activating cells of the cytotoxic T lymphocytes specific to the peptide when they are reinjected into the patient, intradermally, subcutaneously and by intra-ganglion injection.

The fourth generation of vaccine is that of the modified tumour cells.

The melanotic tumour cell bypasses the immune system by producing immunosuppressive cytokines, masking its tumour antigens, and not expressing the co-activation molecules or the antigens of class I or II. The cytotoxic T response is therefore inhibited. The principle of the tumour cell vaccine consists of subcutaneously or intradermally injecting the patient with irradiated autologous tumour cells the phenotypical profile of which has been modified to make it accessible to the cytotoxic T lymphocytes. It is thus that the tumour cells can be transfected by: either IL-7, IL-2 and IL-12 cytokines; or GM-CSF which activates the macrophages; or antigens of class I, II, the antigen BL7 thus allowing the recognition of the tumour antigens by the cytotoxic cell.

The clinical studies (phases I-II) carried out with these different first and second generation vaccines, at the metastatic stage, relate to a limited number of patients with currently an average response level of the order of 20%. These responses are above all obtained on cutaneous, ganglion, pulmonary and hepatic sites. An interesting fact specific to the vaccination is that prolonged response periods (more than 2 years) have been noted. An important point is the duration of the clinical response. In fact, in contrast to the chemotherapies, clinical regression most often appears only after a stabilization phase which can last 3 to 4 months, or even longer.

Tolerance is generally good. The noted side effects being essentially erythema at the injection site, and vitiligo-type auto-immune reactions.

However, the low average response level of the current therapies implies a significant need for new tools making it possible to broaden the range of treatment of malignant melanomas.

It is with the aim of responding to this technical problem that the Applicant has developed a monoclonal antibody, monoclonal antibody fragment or monoclonal antibody derivative, directed against the human LDL (Low Density Lipoprotein) receptor, in which the variable region of each of the light chains is encoded by the murine nucleic acid sequence SEQ ID NO: 5, the variable region of each of the heavy chains is encoded by the murine nucleic acid sequence SEQ ID NO: 7, or by nucleic acid sequences having sufficient homology with the sequences SEQ ID NO: 5 and SEQ ID NO: 7 for the nature and the binding affinity of said antibody to its antigen not to be modified, and in which the constant regions of its light chains and its heavy chains are constant regions originating from a non-murine species.

The antibodies are constituted by heavy chains and light chains, interlinked by disulphide bridges. Each chain is constituted, at the N-terminal position, by a variable region (or domain) (encoded by the rearranged V-J genes for the light chains and V-D-J for the heavy chains) specific to the antigen against which the antibody is directed and, in the C-terminal position, by a constant region constituted by a single LC domain for the light chains or by several domains for the heavy chains.

The two heavy (H) chains and the two light (L) chains are identical to each other. The light chain is made up of 2 domains, a variable domain V and a constant domain C, folded independently of each other in space. These are called VL and CL. The heavy chain also comprises a V domain denoted VH and 3 or 4 C domains denoted CH1 to CH4. Each domain comprises approximately 110 amino acids and is structured in a comparable manner. The 2 heavy chains are linked by disulphide bridges and each heavy chain is also linked to a light chain by a disulphide bridge.

The region which determines the specificity of the antibody for the antigen is borne by the variable parts, whereas the constant parts can interact with the receptors Fc of the effector cells or molecules as complement for mediating different functional properties.

For the purposes of the invention, the expressions “monoclonal antibody” or “monoclonal antibody composition” refer to a preparation of molecules of antibodies possessing an identical and unique specificity. Moreover, throughout the description, claims and figures of the present application, by “monoclonal antibody”, is meant a complete monoclonal antibody, a fragment or a derivative of such an antibody.

Particularly advantageously, within the framework of the invention, sufficient homology (for the nature and the binding affinity of said antibody to its antigen not to be modified) corresponds to 70%-100% homology.

According to the invention, a first nucleic acid having at least 70% homology with a second reference nucleic acid, will possess at least 90%, preferably at least 70%, 80%, 90%, 91%, 92%, 93%, 94%, 95%, 96%, 97%, 97.5%, 98%, 98.3% 98.6%, 99%, 99.6% nucleotide identity with said second reference nucleic acid.

According to the invention, a first polypeptide having at least 70% identity with a second reference polypeptide, will possess at least 90%, preferably at least 70%, 80%, 90%, 91%, 92%, 93%, 94%, 95%, 96%, 97%, 97.5%, 98%, 98.3% 98.6%, 99%, 99.6% amino acid identity with said second reference polypeptide.

The “percentage of homology” between two nucleic acid sequences or between two polypeptide sequences, within the meaning of the present invention, is determined by comparing the two sequences, optimally aligned, through a comparison window.

The part of the nucleotide or amino acid sequence in the comparison window can thus comprise additions or deletions (for example of “gaps”) relative to the reference sequence (which does not comprise these additions or these deletions) in order to obtain an optimum alignment between the two sequences.

The percentage of homology is calculated by determining the number of positions at which an identical nucleic base, or an identical amino acid residue, is observed for the two sequences compared, then by dividing the number of positions at which there is identity between the two nucleic bases, or between the two amino acid residues, by the total number of positions in the comparison window, then by multiplying the result by one hundred in order to obtain the percentage nucleotide identity of the two sequences with each other, or the percentage amino acid identity of the two sequences with each other.

The optimal alignment of the sequences for the comparison can be achieved by computer using known algorithms. Quite preferably, the percentage sequence identity is determined using the CLUSTAL W software (version 1.82), the parameters being fixed as follows: (1) CPU MODE=ClustalW mp; (2) ALIGNMENT=<<full>>; (3) OUTPUT FORMAT=<<aln w/numbers>>; (4) OUTPUT ORDER=<<aligned>>; (5) COLOR ALIGNMENT=<<no>>; (6) KTUP (word size)=<<default>>; (7) WINDOW LENGTH=<<default>>; (8) SCORE TYPE=<<percent>>; (9) TOPDIAG=<<default>>; (10) PAIRGAP=<<default>>; (11) PHYLOGENETIC TREE/TREE TYPE=<<none>>; (12) MATRIX <<default>>; (13) GAP OPEN=<<default>>; (14) END GAPS=<<default>>; (15) GAP EXTENSION <<default>>; (16) GAP DISTANCES=<<default>>; (17) TREE TYPE=<<cladogram>> and (18) TREE GRAP DISTANCES=<<hide>>.

According to the invention, by “the nature and the binding affinity of said antibody to its antigen are not modified” is meant the fact, on the one hand, that the antibody having homology with the antibody in which the variable region of each of the light chains is encoded by the sequence SEQ ID NO: 5, and in which the variable region of each of the heavy chains is encoded by the sequence SEQ ID NO: 7, bind to the same antigen, and on the other hand, that their binding affinity is at least equal to 90% of the binding affinity of the antibody in which the variable region of each of the light chains is encoded by the sequence SEQ ID NO: 5, and in which the variable region of each of the heavy chains is encoded by the sequence SEQ ID NO: 7.

By “nucleic acid sequences having sufficient homology with the sequences SEQ ID NO: 5 and SEQ ID NO: 7 for the nature and the binding affinity of the antibody to its antigen not to be modified” is meant any nucleic acid sequence encoding for a polypeptide, peptide or protein, comprising at least one immunoglobulin domain or fragment, as well as any antibody derivative having sufficient homology with the sequences SEQ ID NO: 5 and SEQ ID NO: 7 for the nature and the binding affinity of the antibody to its antigen not to be modified.

Thus by “immunoglobulin domain” is meant any one of the domains VL, CL, VH, CH1, CH2, CH3, CH4. As the antibody according to the invention may advantageously contain one or more of these domains, all the combinations of the domains previously mentioned form part of the invention.

By “immunoglobulin fragment” is meant one of the fragments chosen from the Fab, Fab′, F(ab′)2, Fc fragments, an scFv or a CDR (Complementarity Determining Region).

The enzymatic digestion of the immunoglobulins by papain produces 2 identical fragments, which are called “Fab fragment” (Fragment Antigen Binding), and an Fc fragment (crystallizable fragment). The Fc fragment is the support of the immunoglobulin effector functions.

By digestion with pepsin, a F(ab′)2 fragment is produced, where the two Fab fragments remain linked by two disulphide bridges, and the Fc fragment is split into several peptides. The F(ab′)2 fragment is formed by two Fab′ fragments, linked by intercatenary disulphide bridges to form a F(ab′)2.

As regards the variable regions of the heavy and light chains, it is noted that the sequence variability is not distributed equally. In fact, the variable regions are constituted, on the one hand, by regions varying very little known as “frameworks” (FR), which are 4 in number (FR1 to FR4) and, on the other hand, by regions in which the variability is extreme: these are “hypervariable” regions, or CDR, which are 3 in number (CDR1 to CDR3).

Thus, the antibody according to the invention being able advantageously to contain one or more of these fragments, all the combinations of the previously mentioned fragments form part of the invention.

In a particular aspect of the invention, the antibody according to the invention contains at least one immunoglobulin domain and at least one immunoglobulin fragment, for example an Fc fragment and one or more variable or hypervariable regions.

Finally, by “antibody derivative” is meant any antibody, this antibody being able to comprise one or more mutations, substitutions, deletions and/or additions of one or more amino acid residues. Such an addition, substitution or deletion can be located at any position in the molecule. In the case where several amino acids have been added, substituted or deleted, any combination of addition, substitution or deletion can be considered, on condition that the resulting antibody still has at least the advantageous properties of the antibody of the invention.

The antibody according to the invention, in which the variable regions of the light and heavy chains, or at least one domain or fragment of these regions, belong to a species different from the constant regions of the light chains and of the heavy chains, is qualified as a “chimeric” antibody.

The murine nucleic acid sequences SEQ ID NO: 5 and SEQ ID NO: 7 code for the variable domain of each of the light chains and the variable domain of each of the heavy chains respectively of the antibody produced by the murine hybridoma C7, available from the ATCC (American Type Culture Collection) under number CRL-1691. This hybridoma produces a murine monoclonal antibody of IgG2b isotype directed against the LDL-R.

The human LDL receptor (LDL-R) is a transmembrane protein of 839 amino acids which comprises three regions: the extra-cellular region (1-768), the transmembrane region (768-790) and the cytoplasmic region (790-839). The extracellular region is divided into two sub-regions: that binding the LDLs (1-322) and the sub-region outside the zone binding the LDLs (323-768).

The murine sequences of the antibody of the invention have been chosen in order to encode the variable regions of the antibody according to the invention, or at least one domain or fragment of these regions, because of the specificity of the murine antibody C7 for the LDL-R antigen. The antibody C7 was produced by immunization with the extracellular domain of the bovine LDL-R. It binds, apart from the bovine LDL-R, to the human LDL-R but it does not cross-react with the LDL-Rs of the rat, mouse, Chinese hamster, rabbit and dog (Beisiegel et al. 1981 J. Biol. Chem. 256, 11923-11931). This property is advantageous as the monoclonal antibody production lines are very often lines originating from these species. Thus, the recombinant antibody can be advantageously produced in a cell line originating from the lines YB2/0, NSO, Sp2/0, CHO, this list not being limitative.

The antibody of the invention also means antibodies possessing CDR regions the peptide sequence of which is chosen from the sequences SEQ ID NO: 6, SEQ ID NO: 8, SEQ ID NO: 9, SEQ ID NO: 15, SEQ ID NO: 16, SEQ ID NO: 17, SEQ ID NO: 22, SEQ ID NO: 24, SEQ ID NO: 25, SEQ ID NO: 26, SEQ ID NO: 27, SEQ ID NO: 28.

These sequences are the sequences of the CDR regions originating from the murine antibody C7, the sequence SEQ ID NO: 6 representing the CDR1 sequence of the light chain of the antibody according to Kabat numbering, the sequence SEQ ID NO: 8 representing the CDR2 sequence of the light chain of the antibody according to Kabat numbering, the sequence SEQ ID NO: 9 representing the CDR3 sequence of the light chain of the antibody according to Kabat numbering, the sequence SEQ ID NO: 15 representing the CDR1 sequence of the light chain of the antibody according to IMGT numbering, the sequence SEQ ID NO: 16 representing the CDR2 sequence of the light chain of the antibody according to IMGT numbering, the sequence SEQ ID NO: 17 representing the CDR3 sequence of the light chain of the antibody according to IMGT numbering, the sequence SEQ ID NO: 22 representing the CDR1 sequence of the heavy chain of the antibody according to Kabat numbering, the sequence SEQ ID NO: 24 representing the CDR2 sequence of the heavy chain of the antibody according to Kabat numbering, the sequence SEQ ID NO: 25 representing the CDR3 sequence of the heavy chain of the antibody according to Kabat numbering, the sequence SEQ ID NO: 26 representing the CDR1 sequence of the heavy chain of the antibody according to IMGT numbering, the sequence SEQ ID NO: 27 representing the CDR2 sequence of the heavy chain of the antibody according to IMGT numbering, the sequence SEQ ID NO: 28 representing the CDR3 sequence of the heavy chain of the antibody according to IMGT numbering.

The invention also includes antibodies in which the variable domain of each of the light chains and the variable domain of each of the heavy chains possess at least 70% homology, or advantageously at least 80%, or also 90% or 99%, with the sequences SEQ ID NO: 5 and SEQ ID NO: 7 respectively, the sequence modifications not modifying the specificity of the antibody. Preferably, these sequence modifications do not reduce its affinity for its target.

The antibody according to the invention also possesses constant regions of its light and heavy chains belonging to a non-murine species. In this respect, all the families and species of non-murine mammals are capable of being used, and in particular humans, monkeys, the muridae (except for mice), the suidae, the bovids, the equids, the felids, the canids, for example, as well as birds.

The antibodies according to the invention can be constructed using the standard recombinant DNA techniques well known to a person skilled in the art, and more particularly using the techniques for construction of “chimeric” antibodies described for example in Morrison et al., Proc. Natl. Acad. Sci. U.S.A., 81, pp. 6851-55 (1984), where recombinant DNA technology is used to replace the constant region of a heavy chain and/or the constant region of a light chain of an antibody originating from a non-human mammal with the corresponding regions of a human immunoglobulin. Such antibodies and their preparation method have also been described in the Patent Application EP 173 494, in the document Neuberger, M. S. et al., Nature 312 (5995): 604-1985), as well as in the document EP 125 023 for example. Methods for producing chimeric antibodies are widely available to a person skilled in the art. For example, the heavy and light chains of the antibody can be expressed separately using a vector for each chain, or be incorporated in a single vector.

An expression vector is a nucleic acid molecule in which the murine nucleic acid sequence encoding for the variable domain of each of the heavy or light chains of the antibody and the nucleic acid sequence, preferably human, encoding for the constant region of each of the heavy or light chains of the antibody have been inserted, in order to introduce them and maintain them in a host cell. It allows the expression of these fragments of foreign nucleic acid in the host cell as it possesses sequences (promoter, polyadenylation sequence, selection gene) indispensable to this expression. The vector can be for example a plasmid, an adenovirus, a retrovirus or a bacteriophage, and the host cell can be any mammal cell, for example SP2/0, YB2/0, IR983F, the human myeloma Namalwa, PER. C6, the CHO lines, in particular CHO—K—I, CHO-Lec10, CHO-Lec1, CHO-Lec13, CHO Pro-5, CHO dhfr-, Wil-2, Jurkat, Vero, Molt-4, COS-7, 293-HEK, BHK, K6H6, NS0, SP2/0-Ag 14 and P3X63Ag8.653.

For the construction of expression vectors of the chimeric antibodies according to the invention, synthetic signal sequences and appropriate restriction sites can be fused to the variable regions during PCR amplification reactions. The variable regions are then combined with the constant regions of an antibody, preferably a human IgG1. The genes thus constructed are cloned in order to place them under the control of a promoter, for example the RSV (Rous Sarcoma Virus), CMV (cytomegalovirus), MLP (Major Late Promoter) promoter, this list not being limitative, and upstream of a polyadenylation site, using two separate vectors (one for each chain). The vectors are also provided with selection genes known to a person skilled in the art, such as for example the dhfr (dihydrofolate reductase) gene or the neomycin resistance gene.

The chimeric antibodies according to the invention can be produced by co-transfection in a host cell of the expression vector of the light chain and of the expression vector of the heavy chain using a method well known to a person skilled in the art (for example co-precipitation with calcium phosphate, electroporation, micro-injection, etc.). At the end of the transfection, the cells can be placed in selective medium, for example in RPMI medium (Invitrogen, ref 21875-034) containing 5% dialysed serum (Invitrogen, ref. 10603-017), 500 μg/ml of G418 (Invitrogen, ref. 10131-027) and 25 nM of methotrexate (Sigma, ref. M8407). The supernatants of the resistant transfection wells are screened for the presence of chimeric immunoglobulin (Ig) by ELISA assay specific to the human Ig sequences or sequences from another species if the constant part comes from another species. The transfectants producing the most antibodies are amplified and their supernatant reassayed by ELISA in order to estimate their productivity and to select the best 3 producers for cloning by limit dilution (40 cells/plate).

A particular embodiment is illustrated below.

Preferably, the variable region each of the light chains of the antibody according to the invention possesses the peptide sequence SEQ ID NO: 10 and the variable region of each of the heavy chains of the antibody according to the invention possesses the peptide sequence SEQ ID NO: 11. The peptide sequence SEQ ID NO: 10 is the peptide sequence deduced from the nucleotide sequence SEQ ID NO: 5 and the peptide sequence SEQ ID NO: 11 is the sequence deduced from the nucleotide sequence SEQ ID NO: 7.

Preferably, the constant regions of each of the light chains and of each of the heavy chains of the antibody according to the invention are human constant regions. This preferred embodiment of the invention makes it possible to reduce the immunogenicity of the antibody in humans and similarly to improve its efficiency during its therapeutic or prophylactic administration to humans.

In a preferred embodiment of the invention, the constant region of each of the light chains of the antibody according to the invention is of type K. Any allotype is suitable for carrying out the invention, for example Km(1), Km(1, 2), Km(1, 2, 3) or Km(3).

In another embodiment of the invention, the constant region of each of the light chains of the antibody according to the invention is of type λ.

In a particular aspect of the invention, and in particular when the constant regions of each of the light chains and of each of the heavy chains of the antibody according to the invention are human regions, the constant region of each of the heavy chains of the antibody is of type γ. According to this variant, the constant region of each of the heavy chains of the antibody can be of type γ1, type γ2, type γ3, these three types of constant regions having the feature of binding the human complement, or also of type γ4. The antibodies possessing a constant region of each of the heavy chains of type γ belong to the IgG class. The type G immunoglobulins (IgG) are heterodimers constituted by two heavy chains and two light chains, interlinked by disulphide bridges. Each chain is constituted, at the N-terminal position, by a variable region or domain (encoded by the rearranged V-J genes for the light chain and V-D-J for the heavy chain) specific to the antigen against which the antibody is directed, and in the C-terminal position, by a constant region, constituted by a single LC domain for the light chains or by 3 domains (CH1, CH2 and CH3) for the heavy chain. The combination of the variable domains V_{H} and V_{L} and constant domains CH₁ and CL of the heavy and light chains form the Fab parts, which are connected to the Fc region by a very flexible hinge region making it possible for each Fab to bind to its antigen target whilst the Fc region, mediating the effector properties of the antibody remains accessible to the effector molecules such as the FcγR receptors, the neonatal Fc receptor (FcRn) and C1q. The Fc region, constituted by the 2 globular domains Cγ₂ and Cγ₃, is glycosylated at the Cγ₂ domain with the presence, on each of the 2 chains, of a biantennate N-glycan, linked to the Asn 297.

Preferably, the constant region of each of the heavy chains of the antibody is of type γ1, as such an antibody shows an ability to produce an ADCC (Antibody-Dependent Cellular Cytotoxicity) activity in most (human) individuals. In this respect, any allotype is suitable for carrying out the invention, for example G1m(3), G1m (1, 2, 17), GIm(1, 17) or G1m(1,3).

In a particular aspect of the invention, the constant region of each of the heavy chains of the antibody is of type γ1, and it is encoded by the human nucleic acid sequence SEQ ID NO: 23, the constant region of each of its light chains being encoded by the human nucleic acid sequence SEQ ID NO: 21, or a sequence homologous to the sequence SEQ ID NO: 23 or SEQ ID NO: 21, or a fragment of said sequences. Thus, such an antibody possesses a murine variable region and a human constant region, with heavy chains of type γ1. This antibody therefore belongs to the IgG1 sub-class. This antibody possesses two light chains the variable domain of which is encoded by the murine nucleic acid sequence SEQ ID NO: 5 and the human constant region is encoded by the nucleic acid sequence SEQ ID NO: 21, and two heavy chains the variable domain of which is encoded by the murine nucleic acid sequence SEQ ID NO: 7 and the constant region is encoded by the human nucleic acid sequence SEQ ID NO: 23.

Preferably, each of the light chains of the antibody according to the invention is encoded by the murine-human chimeric nucleic acid sequence SEQ ID NO: 13, and each of the heavy chains is encoded by the murine-human chimeric nucleic acid sequence SEQ ID NO: 19. The murine-human chimeric nucleic acid sequence SEQ ID NO: 13 encoding for each of the light chains of the antibody is obtained by fusion of the murine nucleic acid sequence SEQ ID NO: 5 encoding for the variable domain of each of the light chains of the antibody and of the human nucleic acid sequence SEQ ID NO: 21 encoding for the constant region of each of the light chains of the antibody. The murine-human chimeric nucleic acid sequence SEQ ID NO: 19 encoding for each of the heavy chains of the antibody is obtained by fusion of the murine nucleic acid sequence SEQ ID NO: 7 encoding for the variable domain of each of the heavy chains of the antibody and of the human nucleic acid sequence SEQ ID NO: 23 encoding for the constant region of each of the heavy chains of the antibody.

In such a particular aspect of the invention, when each of the light chains of the antibody is encoded by the murine-human chimeric nucleic acid sequence SEQ ID NO: 13, and each of the heavy chains is encoded by the murine-human chimeric nucleic acid sequence SEQ ID NO: 19, the peptide sequence of each of the light chains, deduced from the nucleic acid sequence SEQ ID NO: 13, is the sequence SEQ ID NO: 14 and the peptide sequence of each of the heavy chains, deduced from the nucleic acid sequence SEQ ID NO: 19, is the sequence SEQ ID NO: 20.

The invention also relates to antibodies in which each of the light chains encoded by a murine-human chimeric nucleic acid sequence possesses at least 70% homology with the murine-human chimeric nucleic acid sequence SEQ ID NO: 13 and each of the heavy chains encoded by a murine-human chimeric nucleic acid sequence possesses at least 70% homology, or advantageously at least 80%, or also 90% or 99%, with the murine-human chimeric nucleic acid sequence SEQ ID NO: 19, these modifications altering neither the specificity of the antibody nor its effector activities.

Advantageously, the antibody according to the invention is coupled to a toxin. The toxin is, for example, the diphtheria toxin or ricin, this list not being limitative. The bond between the antibody according to the invention and the toxin is sufficiently strong to avoid the systemic release of the toxin and also sufficiently labile for the toxin to be released in the target cells.

In another aspect of the invention, the antibody is coupled to a radio-isotope. The presence of the radio-isotope considerably increases the cytotoxicity. Two isotopes are essentially used: iodine 131 (beta and gamma emitter), the half-life of which is relatively long (8 days) and which exerts a tumoricidal effect over approximately 1 mm around the tumour cell having bound the antibody according to the invention. Iodine 131 has the advantage of making imaging possible, but requires compliance with radiation-protection measures. Yttrium 90 (beta emitter), the half-life of which is shorter (2.5 days), exerts tumoricidal effects over a distance of 5 mm.

Advantageously, the antibody of the invention is produced in a rat hybridoma cell line. The antibody producing line according to the invention is an important characteristic since it confers on the antibody certain of its particular properties. In fact, the means of expression of the antibodies is at the origin of the post-translational modifications, in particular glycosylation modifications, which can vary from one cell line to the other, and thus confer different functional properties on antibodies which however have identical primary sequences.

In a certain embodiment, the antibody is produced in the rat line YB2/0 (cell YB2/3HL.P2.G11.16Ag.2O, deposited at the American Type Culture Collection under number ATCC CRL-1662).

A preferred antibody according to the invention is the antibody EMAB604 produced by the hybridoma R604 (deposited on 14 Nov. 2006 under number I-3692 at the CNCM—Institut Pasteur, 25 rue du Professeur Roux, 75724 Paris Cedex 15, France). The variable region of each of the light chains of the monoclonal antibody produced by the hybridoma R604 is encoded by the nucleic acid sequence SEQ ID NO: 5, and the variable region of each of the heavy chains of the monoclonal antibody produced by the hybridoma R604 is encoded by the nucleic acid sequence SEQ ID NO: 7.

A particular subject of the invention relates to a monoclonal antibody binding to the LDL-R and allowing the recruitment of effector cells. Preferably, this antibody is EMAB604, or any humanized or human chimeric antibody possessing functional characteristics identical to the antibody EMAB604. Advantageously, this antibody is produced by the cell line R604.

Another subject of the invention relates to the expression vector of the light chain of an antibody according to the invention, of sequence SEQ ID NO: 12. This vector is the vector allowing the expression of an antibody according to the invention the light chain of which is encoded by the nucleic acid sequence SEQ ID NO: 13, the deduced peptide sequence of which is the sequence SEQ ID NO: 14. This vector is a nucleic acid molecule into which the murine nucleic acid sequence SEQ ID NO: 5 encoding for the variable domain of each of the light chains of the antibody and the nucleic acid sequence SEQ ID NO: 21 encoding for the constant region of each of the light chains of the antibody have been inserted, in order to introduce them into and maintain them in a host cell. It allows the expression of these fragments of foreign nucleic acid in the host cell as it possesses sequences (promoter, polyadenylation sequence, selection gene) indispensable to this expression. Such vectors are well known to a person skilled in the art, and can be an adenovirus, a retrovirus, a plasmid or a bacteriophage, this list not being limitative. Furthermore, any mammal cell can be used as host cell, i.e. as a cell expressing the antibody according to the invention, for example YB2/0, CHO, CHO dhfr- (for example CHO DX B11, CHO DG44), CHO Lec13, SP2/0, NSO, 293, BHK or COS.

Another subject of the invention relates to the expression vector of the heavy chain of an antibody according to the invention, of sequence SEQ ID NO: 18. This vector is the vector allowing the expression of an antibody according to the invention the heavy chain of which is encoded by the nucleic acid sequence SED ID NO: 19, the deduced peptide sequence of which is the sequence SEQ ID NO: 20. This vector is a nucleic acid molecule into which the murine nucleic acid sequence SEQ ID NO: 7 encoding for the variable domain of each of the heavy chains of the antibody and the human nucleic acid sequence SEQ ID NO: 23 encoding for the constant region of each of the heavy chains of the antibody have been inserted, in order to introduce them into and maintain them in a host cell. It allows the expression of these fragments of foreign nucleic acid in the host cell as it possesses sequences (promoter, polyadenylation sequence, selection gene) indispensable to this expression. As indicated previously, the vector can be for example a plasmid, an adenovirus, a retrovirus or a bacteriophage, and the host cell can be any mammal cell, for example YB2/0, CHO, CHO dhfr- (CHO DX B11, CHO DG44), CHO Lec13, SP2/0, NSO, 293, BHK or COS. An antibody produced by co-expression of these vectors in the cell YB2/0 is illustrated by the anti-LDL-R antibody EMAB604, produced by the clone R604 (deposited under registration number CNCM I-3692 at the CNCM). This antibody possesses a strong cytotoxic activity, determined by an ADCC test. Furthermore, the antibody EMAB604 induces the secretion of IL-2 by the Jurkat-CD16 cell line, this test being used to demonstrate the ability of the CD16 receptor to be activated by the antibodies. The antibody EMAB604, which can be produced by culture of the clone R604 in a culture medium and under conditions allowing the expression of the vectors previously described, is therefore one of the most useful tools capable of advancing the therapy and the diagnosis of melanomas.

Another subject of the invention relates to a stable cell line producing an antibody according to the invention as described previously.

Advantageously, the stable cell line according to the invention, which is characterized in that the cell line in which the antibody is expressed, is chosen from the group consisting of: SP2/0, YB2/0 (cell YB2/3HL.P2.G11.16Ag.20, deposited at the American Type Culture Collection under number ATCC CRL-1662), SP2/0-AG14 (ATCC CRL-1581), IR983F, the human myeloma Namalwa, PERC6, the CHO lines, in particular CHO—K-1, CHO-Lec1O, CHO-Lec1, CHO-Lec13, CHO Pro-5, CHO dhfr-, Wil-2, Jurkat, Vero, Molt-4, COS-7, 293-HEK, BHK, K6H6, NSO, SP2/0-Ag 14 and P3X63Ag8.653.

The stable cell line of the invention has incorporated the two expression vectors described previously.

Another subject of the invention relates to the hybridoma R604 deposited under registration number CNCM I-3692 at the Collection Nationale de Cultures de Microorganismes (CNCM, Institut Pasteur, 25 rue du Docteur Roux, 75724 Paris Cedex 15).

Advantageously, the antibody according to the invention allows the recruitment of effector immune cells.

Such an antibody, because of its good specificity and its good affinity, is a tool which can serve to mediate ADCC (Antibody Dependent Cellular-mediated Cytotoxicity) reactions. In fact, the antibody according to the invention has a good affinity for the LDL-R and on the other hand, allows the recruitment of effector immune cells. For the purposes of the invention, by “effector immune cell” is meant a cell which causes the destruction of the cells to which the antibody according to the invention is bound (“target cells”).

The anti-LDL-R antibody EMAB604 has the ability to interact strongly with the Fcgamma receptor RIIIa or CD16 expressed by the NK cells. This binding is at least three times greater than that of the anti-CD20 antibody Rituxan® and comparable to that of the anti-CD20 antibody produced by the EMABLing platform of LFB. This strong binding to the CD16 receptor makes it possible to envisage optimized cytotoxic capacities for the anti-LDL-R antibody EMAB604.

Thus it has been shown that this antibody induces cell lysis by ADCC (Antibody-Dependent Cell-mediated Cytotoxicity) of the HT144 (melanoma) cells and GUY 17.2 cells dependent on the interaction of its Fc part with the low affinity CD16 receptor expressed on the NK (Natural Killer) cells. In fact, this ADCC is inhibited in the presence of an anti-CD16 antibody (clone 3G8). It should be noted that CD16 is also expressed by macrophages and in a sub-population of monocytes which makes it possible to envisage the action of the anti-LDL-R antibody EMAB604 by induction of a cell cytotoxicity via cells of the monocyte line.

Furthermore, this strong binding of the anti-LDL-R antibody EMAB604 to the CD16 receptor confers upon it, in the presence of HT144 cells, the ability to induce the secretion of interleukin-2 (IL-2) by the Jurkat line transfected with the CD16 (Jurkat-CD16). In fact, the engagement of the Fc part of the antibody bound to its target (HT144) induces an activation signal which results in the secretion of IL-2 by Jurkat-CD16.

The antibody C7 was obtained by immunization of mice with the partially purified bovine LDL-R. It has been shown that it is a good LDL competitor, and therefore has an affinity for the LDL-R comparable to that of the natural LDL-R ligand.

These antibodies bind to the target cell by their variable fragment and bind to the effector cells by their constant fragment. This dependent relation of the antibodies between the target cells and the effector cells causes the lysis of the target cells by a mechanism of ADCC (Antibody Dependent Cellular Cytotoxicity) type.

Advantageously, the target cells according to the invention are tumour cells such as sarcomas, myelomas, melanomas, lymphomas and leukemias, this list not being limitative. In fact, studies have shown a correlation between the increase in the level of expression of the LDL-R by the cells and certain cancers. It turns out that patients suffering from certain cancers have hypocholesterolemia. This hypocholesterolemia is the consequence of an over-use of cholesterol by the cancer cells. For their survival, the latter induce an increase in the level of expression of the LDL receptor (LDL-R) within the tumorous organs (Henricksson et al., 1989). There can be mentioned in particular cancer of the prostate, breast, liver, pancreas, ovaries, colon, lung and stomach and leukemias.

As a result, the cancer cells over-expressing the LDL-R are therefore preferred targets of the antibody according to the invention. It has thus been shown that the anti-LDL-R antibody EMAB604 binds to the melanoma lines HT144 and GUY 17.2.

Thus, the invention relates to a monoclonal antibody directed against the human LDL (Low Density Lipoprotein) receptor, capable of inducing a lysis specific to the melanomas.

Another subject of the invention is the use of an antibody according to the invention, in order to activate in vitro, ex vivo or in vivo, the FcγRIII receptors of effector immune cells or to cause the secretion of cytokines or chemokines by effector cells. In fact, the antibodies of the invention can be used for their ability to activate the FcγRIIIA receptor by their Fc region. This represents a considerable benefit, as this receptor is expressed at the surface of cells called “effector cells”: the binding of the Fc region of the antibody to its receptor borne by the effector cell causes the activation of the FcγRIIIA and the destruction of the target cells. The effector cells are for example NK (Natural Killer) cells, macrophages, neutrophiles, the CD8 lymphocytes, the Tγδ lymphocytes, NKT cells, eosinophiles, basophiles or mastocytes.

Another particular subject of the invention is an antibody as described previously for its use as a medicament.

In a particular aspect of the invention, the antibody used binds to the human LDL receptor, and allows the recruitment of effector cells.

Another subject of the invention is the use of a monoclonal antibody directed against the human LDL receptor for the production of a medicament intended for the treatment of cancer, such as cancer of the prostate, breast, liver, pancreas, ovaries, colon, lung, stomach and the leukemias. In fact, the antibody according to the invention specifically targets the LDL-R. In this respect, the antibody according to the invention, by binding to this receptor, will give rise to a reaction of lysis of the target cancer cells, in particular by ADCC against the target cancer cells and allow the lysis of the latter. Thus, the lysed cells are quasi-specifically cancer cells, healthy cells not overexpressing or only slightly expressing the LDL-R and thus being preserved.

Advantageously, the cancers treated by means of the antibody according to the invention are the cancers for which the LDL receptor is overexpressed at the surface of the cancer cells, compared with corresponding healthy cells.

Particularly advantageously, the cancer treated is a melanoma.

Thus, the invention also relates to the use of a monoclonal antibody directed against the human LDL receptor, or of an antibody as defined above, for the production of a medicament intended for the treatment of melanoma. The target cancer cells can be lysed by the effector cells recruited during the ADCC reaction, healthy cells expressing little or no LDL-R or being, if appropriate, treated beforehand in order to induce the internalization of the LDL-R in order not to be recognized by the antibody and thus be preserved.

The malignant melanomas treated can be superficial spreading melanomas, which have the appearance of multi-coloured patches, the contours and surface of which are irregular, and which correspond to the horizontal development phase of malignant melanoma, and nodular melanomas characterized clinically by a protruding melanotic tumour and histologically by a well-circumscribed proliferation which immediately invades the dermis.

Advantageously, the antibody of the invention is capable of inducing a lysis specific to melanomas originating from the lines HT144 (HTB-63) (HLA Al, Aw24, B13, B15, Cw3, DRw4, DRw7) and GUY-17.2, dependent on the CD16.

Another subject of the invention relates to the use of an antibody of the invention in combination with one or more other antibodies directed against one or more other antigens expressed on the melanoma cells. Such antigens can be expressed on the lymphoid cells and are chosen from HLA-DR, CD20, CD22, CD23, CD25, CD30, CD33 and CD40.

Another subject of the invention relates to the use of an antibody of the invention in combination with one or more other medicaments commonly used for the treatment of cancers such as cytotoxic and cytostatic medicaments (chemotherapy). These medicaments are well known to a person skilled in the art, among which there can be mentioned, for the cytotoxic agents, alkylating agents and nitrogen mustards, platinum derivatives, intercalating agents, antimetabolites, topoisomerase inhibitors, spindle poisons, and, for the cytostatic agents, tyrosine kinase receptor inhibitors, in particular EGF (endothelial growth factor) receptors, insulin receptors, PDGF (Platelet-derived growth factor) receptors, FGF (fibroblast growth factor) receptors and VEGF (vascular endothelial growth factor) receptors, this list not being limitative.

In a particular aspect of the invention, the use of the antibodies of the invention is carried out in combination, in vitro, ex vivo or in vivo, with cells expressing FcγR, such as the NK (Natural Killer) cells, NKT (Natural Killer T) cells, Tγδ lymphocytes, macrophages, monocytes, any cell genetically modified in order to express CD16, or dendritic cells.

Another subject of the invention relates to a pharmaceutical composition comprising at least one antibody according to the invention as described above and a pharmaceutically acceptable excipient and/or vehicle. This pharmaceutical composition is intended to target the cancer cells, in particular those overexpressing the LDL-R. These cancer cells expressing at their surface a quantity of LDL receptors greater than the quantity of receptors expressed by the healthy cells, the medicament thus prepared is preferentially bound by the cancer cells.

The excipient can be any solution, such as a saline, physiological, isotonic, buffered solution etc., as well as any suspension, gel, powder, etc., compatible with a pharmaceutical use and known to a person skilled in the art. The compositions according to the invention can also contain one or more agents or vehicles chosen from the dispersing agents, solubilizing agents, stabilizers, surfactants, preservatives, etc.

Advantageously, the composition of the invention also comprises at least one antibody directed against another antigen present on the target cells of the antibodies.

Advantageously, the composition of the invention also comprises an anti-HLA-DR antibody. In fact, the melanoma lines HT144 and GUY 17.2 express the HLA-DR antigen at their surface. A composition comprising a mixture of anti-LDL-R and anti-HLA DR antibodies in proportions which can represent 5, 25, 50, 75 or 95% of either antibody with respect to the total weight of the composition is particularly advantageous for the treatment of patients suffering from melanomas.

Moreover, the compositions can be administered in different ways and in different forms. The administration can be carried out by any standard route for this type of therapeutic approach, in particular by systemic route, in particular by intravenous, intradermal, intratumoral, subcutaneous, intraperitoneal, intramuscular, intra-arterial injection, etc. There can be mentioned for example intratumoral injection or injection into an area close to the tumour or irrigating the tumour. Administration can also be oral, mucosal or topical.

The doses can vary depending on the number of administrations, combination with other active ingredients, on the stage of development of the pathology, etc.

Another subject of the invention is the use of the antibody according to the invention in the immunohistochemical analyses of cancerous, healthy or cirrhotic tissues, or in Western Blot, or ELISA analyses or in vivo, ex vivo or in vitro quantification tests.

Other aspects and advantages of the invention are described in the examples which follow, which should be considered as illustrative and do not limit the scope of the invention.

### FIGURES

FIG. 1: ADCC activity of the anti-LDL-R (EMAB604) and anti-HLA-DR CHO antibodies on the melanoma line GUY 17.2. The results are expressed as a percentage of lysis (average±SEM, n=2) depending on the antibody concentration.

FIG. 2: Inhibition of the ADCC activity induced by 10 μg/ml of the anti-LDL-R (EMAB604) and anti-HLA-DR-CHO antibodies on the melanoma line GUY-17.2 by the murine anti-CD16 antibody 3G8 (250 μg/ml).

FIG. 3: ADCC activity of the anti-LDL-R (EMAB604) and anti-HLA-DR CHO antibodies on the melanoma line HT-144. The results are expressed as percentage of lysis (average±SEM, n=2) depending on the antibody concentration.

FIG. 4: Inhibition of the ADCC activity induced by 10 μg/ml of the anti-LDL-R (EMAB604) and anti-HLA-DR-CHO antibodies on the melanoma line HT-144 by the murine anti-CD16 antibody 3G8 (250 μg/ml).

FIG. 5: Secretion of IL-2 by the Jurkat-CD16 cell induced by the anti-LDL-R (EMAB604) and anti-HLA-DR CHO antibodies in the presence of the melanoma line GUY 17.2.

FIG. 6: Secretion of IL-2 by the Jurkat-CD16 cell induced by the anti-LDL-R (EMAB604) and anti-HLA-DR CHO antibodies in the presence of the melanoma line HT-144.

FIG. 7: Percentage of binding of the anti-LDL-R (EMAB604), anti-CD20 antibodies produced in YB2/0 and Rituxan® on the CD16 receptor expressed by the NK cells. The results are expressed for a concentration of 10 and 50 μg/ml of antibody.

FIG. 8: Sequences:

SEQ ID NO: 5: murine nucleic acid sequence encoding for the variable region of each of the light chains of the antibody,

SEQ ID NO: 6: CDR1 sequence of the light chain of the antibody according to Kabat numbering,

SEQ ID NO: 7: murine nucleic acid sequence encoding for the variable region of each of the heavy chains of the antibody,

SEQ ID NO: 8: CDR2 sequence of the light chain of the antibody according to Kabat numbering,

SEQ ID NO: 9: CDR3 sequence of the light chain of the antibody according to Kabat numbering,

SEQ ID NO: 10: peptide sequence of the variable region of each of the light chains of the antibody,

SEQ ID NO: 11: peptide sequence of the variable region of each of the heavy chains of the antibody,

SEQ ID NO: 12: nucleic acid sequence corresponding to the expression vector of the light chain of the antibody,

SEQ ID NO: 13: murine-human chimeric nucleic acid sequence encoding for each of the light chains of the antibody,

SEQ ID NO: 14: peptide sequence of each of the light chains of the antibody deduced from the nucleic acid sequence SEQ ID NO: 13,

SEQ ID NO: 15: CDR1 sequence of the light chain of the antibody according to IMGT numbering,

SEQ ID NO: 16: CDR2 sequence of the light chain of the antibody according to IMGT numbering,

SEQ ID NO: 17: CDR3 sequence of the light chain of the antibody according to IMGT numbering,

SEQ ID NO: 18: nucleic acid sequence corresponding to the expression vector of the heavy chain of the antibody according to the invention,

SED ID NO: 19: nucleic acid sequence encoding for the heavy chain of the antibody according to the invention,

SEQ ID NO: 20: peptide sequence of the heavy chain of the antibody deduced from the sequence SEQ ID NO: 19,

SEQ ID NO: 21: nucleic acid sequence encoding for the constant region of each of the light chains of the antibody,

SEQ ID NO: 22: CDR1 sequence of the heavy chain of the antibody according to Kabat numbering,

SEQ ID NO: 23: human nucleic acid sequence encoding for the constant region of each of the heavy chains of the antibody of γ1 type,

SEQ ID NO: 24: CDR2 sequence of the heavy chain of the antibody according to Kabat numbering,

SEQ ID NO: 25: CDR3 sequence of the heavy chain of the antibody according to Kabat numbering,

SEQ ID NO: 26: CDR1 sequence of the heavy chain of the antibody according to IMGT numbering,

SEQ ID NO: 27: CDR2 sequence of the heavy chain of the antibody according to IMGT numbering,

SEQ ID NO: 28: CDR3 sequence of the heavy chain of the antibody according to IMGT numbering.

SEQ ID NO: 31: peptide sequence of the constant region of each of the light chains of the antibody, deduced from SEQ ID NO: 21, and

SEQ ID NO: 34: peptide sequence of the constant region of each of the heavy chains of the antibody deduced from SEQ ID NO: 23.

### EXAMPLES

### Example 1

### Construction of the Expression Vectors of the Anti-LDL-R Chimeric Antibody C7

### A. Determination of the Leader Sequence of the Variable Regions of the Murine Antibody C7

The total RNA of the murine hybridoma C7 producing an immunoglobulin of type IgG2b,κ was extracted (Macherey-Nagel Nucleospin RNA kit ref. 740609.250). After reverse transcription, the variable domains of the light (Vκ) and heavy (VH) chains of the antibody C7 were amplified by the 5′RACE (Rapid Amplification of cDNA Ends) technique (5′RACE kit, Invitrogen ref. 18374.041).

Briefly, a first reverse transcription stage was first carried out using a primer located in the 5′ region of the murine Cκ or γ2b constant regions. A poly-dC sequence was then added at the 3′ position of the synthesized cDNAs before carrying out the amplification of the Vκ and VH regions using a 5′ primer recognizing the poly-dC sequence, and a 3′ primer located in the murine Cκ or γ2b constant regions at the 5′ position of the reverse transcription primer. The primers used for these two stages are as follows:

1. reverse transcription primers
a. Murine Kappa specific antisense primer

| | |
|---|---|
| 5′-ACT GCC ATC AAT CTT CCA CTT | (SEQ ID NO: 1) |
| GAC-3′ | |

b. Murine γ2b specific antisense primer

| | |
|---|---|
| 5′-GTGTAGAGTCCAGACTGCAGGAG-3′ | (SEQ ID NO: 2) |

2. 5′ RACE PCR primers
a. Murine Kappa specific antisense primer

| | |
|---|---|
| 5′-TTGTTCAAGAAGCACACGACTGAGGCAC-3′ | (SEQ ID NO: 3) |

b. Murine γ2b specific antisense primer

| | |
|---|---|
| 5′-CACTGACTCAGGGAAGTAGCCCTTG-3′ | (SEQ ID NO: 4) |

The VH and Vκ PCR products thus obtained were cloned into the vector pCR4 Blunt-TOPO (Zero blunt TOPO PCR cloning kit, Invitrogen, ref. K2875-20) then sequenced.

The nucleotide sequence of the Vκ region of the murine antibody C7 is indicated under the sequence SEQ ID NO: 5 and the deduced peptide sequence is the sequence SEQ ID NO: 10. The Vκ gene belongs to the Vκ1 sub-group [Almagro J C et al. Immunogenetics (1998), 47: 355-363]. The CDR1, CDR2 and CDR3 sequences of the Vκ region of the murine antibody C7, defined according to Kabat numbering [Kabat et al., “Sequences of Proteins of Immunological Interest”, NIH Publication, 91-3242 (1991)], are indicated under the following sequences: SEQ ID NO: 6, SEQ ID NO: 8 and SEQ ID NO: 9, respectively. The CDR1-IMGT, CDR2-IMGT and CDR3-IMGT sequences of the Vκ region of the murine antibody C7, defined according to IMGT (international ImMunoGeneTics database) analysis [Lefranc, M.-P. et al., Dev. Comp. Immunol., 27, 55-77 (2003)] are indicated under the following sequences: SEQ ID NO: 15, SEQ ID NO: 16 and SEQ ID NO: 17, respectively. This definition, unlike that of Kabat which is based solely on sequence variability analysis, takes into account and combines the characterization of the hypervariable loops [Chothia C. and Lesk A. M. J. Mol. Biol. 196: 901-17 (1987)] and the structural analysis of the antibodies by crystallography.

The nucleotide sequence of the VH region of C7 is the sequence SEQ ID NO: 7 and the peptide sequence which is deduced from it is the sequence SEQ ID NO: 11. The VH gene belongs to the sub-group VH1 [Honjo T. and Matsuda F. in “Immunoglobulin genes”. Honjo T. and Alt F. W. eds, Academic Press, London (1996), pp 145-171]. The CDR1, CDR2 and CDR3 sequences of the VH region of the murine antibody C7, defined according to Kabat numbering [Kabat et al., “Sequences of Proteins of Immunological Interest”, NIH Publication, 91-3242 (1991)], are indicated under the following sequences: SEQ ID NO: 22, SEQ ID NO: 24 and SEQ ID NO: 25, respectively. The CDR1-IMGT, CDR2-IMGT and CDR3-IMGT sequences of the VH region of the murine antibody C7, defined according to IMGT (international ImMunoGeneTics database) analysis [Lefranc, M.-P. et al., Dev. Comp. Immunol., 27, 55-77 (2003)] are indicated under the following sequences: SEQ ID NO: 26, SEQ ID NO: 27 and SEQ ID NO: 28, respectively. This definition, unlike that of Kabat which is based solely on sequence variability analysis, takes into account and combines the characterization of the hypervariable loops [Chothia C. and Lesk A. M. J. Mol. Biol. 196: 901-17 (1987)] and the structural analysis of the antibodies by crystallography.

### B. Construction of the Heavy Chain and Light Chain Expression Vectors of the Chimeric Antibody EMAB604

1. Kappa Light Chain Vector

The Vκ (sequence cloned into the pCR4Blunt-TOPO sequencing vector was amplified using the following cloning primers:

a) V_{K} sense primer

(SEQ ID NO: 29)

5′-ATCGAACTAGTGCCGCCACCATGAAGTTGCCTGTTAGGCT-3′

The underlined sequence corresponds to the Spe I restriction site, the sequence in bold type corresponds to a Kozak consensus sequence, the initiator ATG is in italics.

b) V_{K} antisense primer

(SEQ ID NO: 30)

5′-GATGAAGACACTTGGTGCAGCCACAGTTCGTTTGATTTCCAGCTTGG
TGCCT-3′

This primer produces the junction of the murine Vκ sequences (in italics) and human constant region (Cκ) (in bold type). The underlined sequence corresponds to the Dra III restriction site.

The Vκ (PCR product thus obtained contains the sequence encoding the natural peptide signal of the murine antibody C7. This Vκ (PCR was then cloned between the Spe I and Dra III sites of the light chain chimerization vector at the 5′ position of the human Cκ constant region, the nucleic sequence of which is the sequence SEQ ID NO: 21 and the deduced peptide sequence is the sequence SEQ ID NO: 31. The human Cκ (sequence of this chimerization vector was modified beforehand by silent mutagenesis in order to create a Dra III restriction site in order to allow the cloning of murine Vκ (sequences. This chimerization vector contains an RSV promoter and a bGH (bovine Growth Hormone) polyadenylation sequence as well as the dhfr (dihydrofolate reductase) selection gene.

The sequence of the light chain of the chimeric antibody EMAB604 encoded by this vector is presented in SEQ ID NO: 13 for the nucleotide sequence and corresponds to the deduced peptide sequence SEQ ID NO: 14.

2. Heavy Chain Vector

A similar approach was applied for the chimerization of the heavy chain of the antibody EMAB604.

The VH sequence cloned into the pCR4Blunt-TOPO vector was first amplified using the following cloning primers:

a) VH sense primer

(SEQ ID NO: 32)

5′-ATCGAGCTAGCGCCGCCACCATGGAATGGCCTTGTATCTT-3′

The underlined sequence corresponds to the Nhe I restriction site, the sequence in bold type corresponds to a Kozak consensus sequence, the initiator ATG is in italics.

b) VH antisense primer

(SEQ ID NO: 33)

5′-ACCGATGGGCCCTTGGTGGAGGCTGAGGAGACTGTGAGAGT-3′

This primer produces the junction of the murine VH sequences (in italics) and human G1 constant region (in bold type).

The underlined sequence corresponds to the Apa I restriction site.

The amplified VH fragment contains the sequence encoding the natural peptide signal of the murine antibody C7. This VH PCR amplification product was then cloned between the Spe I and Apa I sites of the chimerization vector containing the heavy chain at the 5′ position of the human γ1 constant region the nucleic sequence of which is the sequence SEQ ID NO: 23 and the deduced peptide sequence is the sequence SEQ ID NO: 34. This chimerization vector contains an RSV promoter and a bGH (bovine Growth Hormone) polyadenylation sequence as well as the neo selection gene.

The sequence of the heavy chain of the chimeric antibody EMAB604 encoded by this vector is presented in SEQ ID NO: 19 for the nucleotide sequence and in sequence SEQ ID NO: 20 for the deduced peptide sequence.

### Example 2

### Creation of a Cell Line Derived from the Line YB2/0 Which Produces the Chimeric Antibody EMAB604

The rat line YB2/0 (ATCC #CRL-1662) was cultured in EMS medium (Invitrogen, ref. 041-95181M) containing 5% foetal calf serum (JRH Biosciences, ref. 12103-78P). For the transfection, 5 million cells were electroporated (Bio-Rad electroporator, model 1652077) in ElectroBuffer medium (Cell Projects, ref. EB-110) with 25 μg of light chain vector K463-26-C7 linearized by Avi II, and 26.7 μg of heavy chain vector H-463-27-C7 linearized by Not I. The electroporation conditions applied were 230 volts and 960 microfarads for a 0.5 ml cuvette with a width of 0.4 cm. The contents of the electroporation cuvette were then distributed over 5 96-well plates with a density of 5000 cells/well.

The placing of the cells in selective RPMI medium (Invitrogen, ref 21875-034) containing 5% dialysed serum (Invitrogen, ref. 10603-017), 500 μg/ml of G418 (Invitrogen, ref. 10131-027) and 25 nM of methotrexate (Sigma, ref. M8407), was carried out 3 days after the transfection.

The supernatants of the resistant transfection wells were screened for the presence of chimeric immunoglobulin (Ig) by ELISA assay specific to the human Ig sequences. The 16 transfectants producing the most antibodies were amplified in 24-well plates and were evaluated for their production ability (productivity and maximum production) and for the fucose level of the IgGs produced.

The cloide R604 CH10 (productivity: 8.1 pcd, maximum production 38.5 μg/ml, fucose level of the IgGs 25.5-26.80), hereafter called “R604”, was selected for the production of the chimeric antibody EMAB604.

The production of the chimeric antibody EMAB604 was carried out by expansion of the culture in EMS medium containing 5% bovine Ig-depleted serum (Invitrogen, ref. 16250-078) and 500 μg/ml of G418 (Invitrogen, ref. 10131-027), obtained by dilution to 2×10⁵ cells/ml in 25 cm², 75 cm² and 175 cm² flasks then in a roller bottle. Having reached the maximum volume (0.91), culture was continued until the cell viability was less than 50%. After production, the chimeric antibody EMAB604 was purified by affinity chromatography on protein A and checked by polyacrylamide gel electrophoresis.

### Example 3

### Protocol for Implementation of the ADCC Test

The target cells (HT-144 or GUY 17.2 lines) are incubated with different antibody concentrations (0, 0.1, 1, 10 μg/ml) and the effector cells (NK cells) purified with a negative depletion kit (NK Cell Isolation Kit, Myltenyi, Paris, France) starting with peripheral blood from healthy donors. After incubation for 4 hours, the cytotoxic activity induced by the antibody is measured by colorimetry by assaying in the supernatants, the activity of the enzyme lactate dehydrogenase (LDH) released by the lysed cells. The results are expressed as a percentage of specific lysis depending on the antibody concentration. The Emax values (maximum lysis percentage), as well as the EC50 values (quantity of antibodies inducing 50% maximum lysis), are calculated using the PRISM software (Graphpad Software).

### Example 4

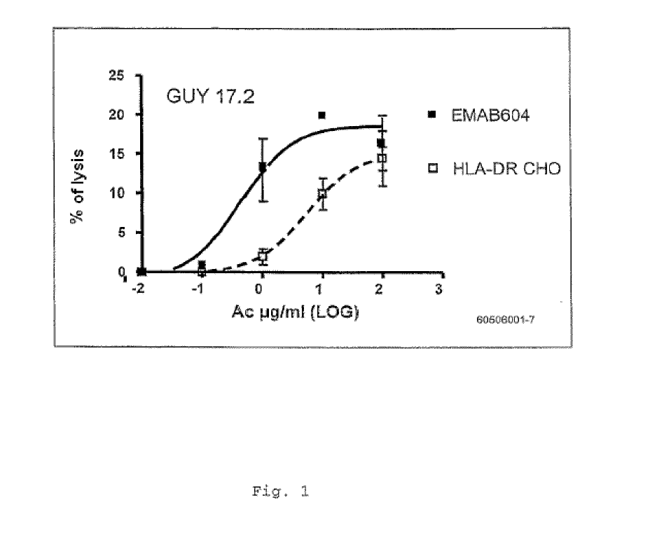

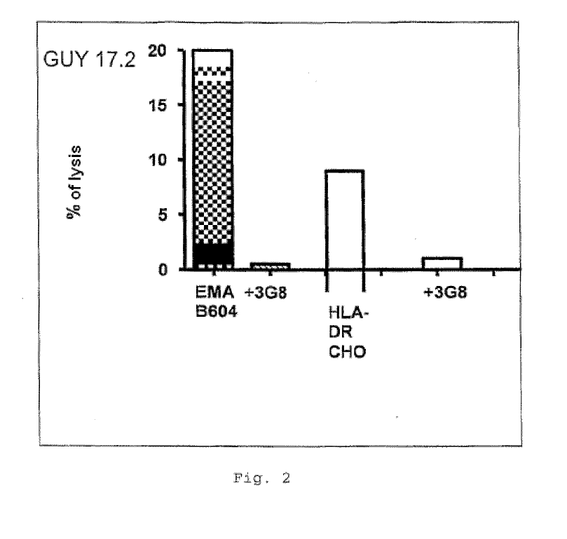

### ADCC Activity of the EMAB604 and Anti-HLA-DR Antibodies Produced by the CHO Line on the Melanoma GUY 17.2 Line (FIG. 1)

The antibody EMAB604 induces a lysis specific to the line GUY-17.2 greater than that induced by an anti-HLA-DR antibody produced in CHO.

The maximum lysis values (Emax) are 19 and 15% for the antibodies EMAB604 and anti-HLA-DR CHO. The corresponding EC50s (quantity of antibodies required to reach 50% maximum lysis) are 0.45 and 5.71 μg/ml respectively, showing that the activity of the antibody EMAB604 is approximately 10 times stronger than that of the anti-HLA-DR antibody produced in CHO.

| | | anti-HLA-DR |
|---|---|---|
| | EMAB604 | CHO |
| Emax | 19 | 15 |
| EC50 | 0.45 | 5.71 |
| (μm/ml) | | |

Calculation of the ADCC values Emax (maximum lysis) and EC50 (antibody concentration required in order to obtain 50% of the Emax) obtained after (sigmoid) modelling of the curve by the PRISM software.

The ADCC activity is dependent on the CD16 since it is inhibited for all the antibodies in the presence of the murine antibody 3G8 (anti-CD16) (FIG. 2).

### Example 5

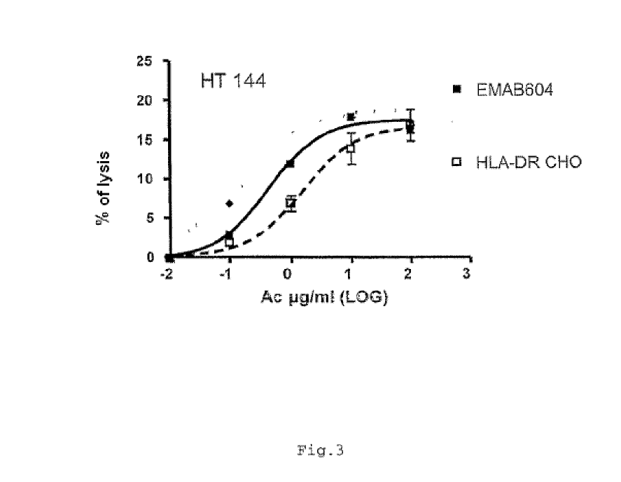

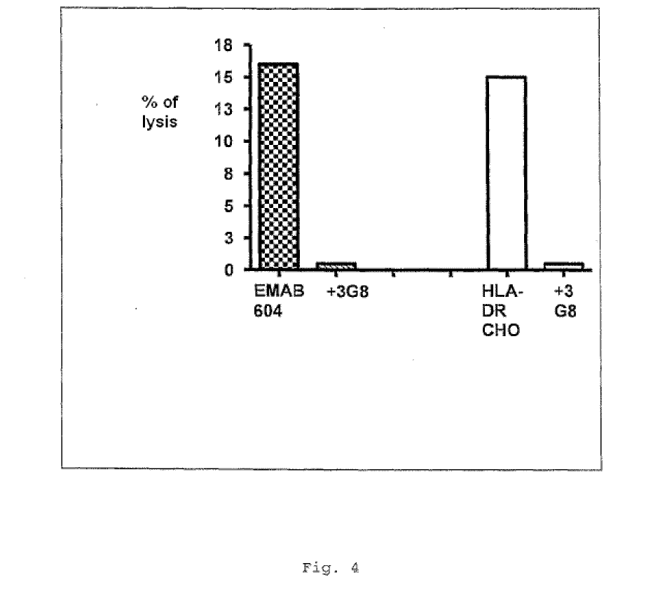

### ADCC Activity of the EMAB604 and Anti-HLA-DR Antibodies Produced by the CHO Line on the Melanoma Line HT-144 (FIG. 3)

The antibody EMAB604 of the invention induces a lysis specific to the melanoma line HT-144 greater than that induced by an anti-HLA-DR antibody produced in CHO.

The maximum lysis values (Emax) are 18 and 17% for the EMAB604 and Anti-HLA-DR CHO antibodies. The corresponding EC50s are 0.45 and 1.45 μg/ml respectively, showing that the activity of the antibody EMAB604 is approximately 8 times stronger than that of the anti-HLA-DR antibody produced in CHO.

| | | anti-HLA-DR |
|---|---|---|
| | EMAB604 | CHO |
| Emax | 18 | 17 |
| EC50 | 0.45 | 1.45 |
| (μm/ml) | | |

Calculation of the Emax ADCC values (maximum lysis) and EC50 (antibody concentration required in order to obtain 50% of the Emax) obtained after (sigmoid) modelling of the curve by the PRISM software.

The ADCC activity is dependent on the CD16 since it is inhibited for all the antibodies in the presence of the murine antibody 3G8 (anti-CD16) (FIG. 4).

### Example 6

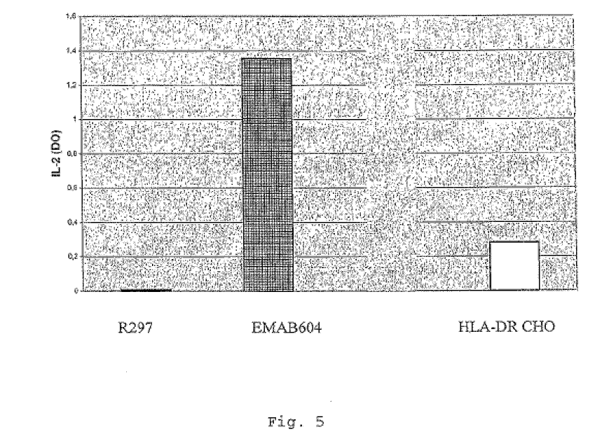

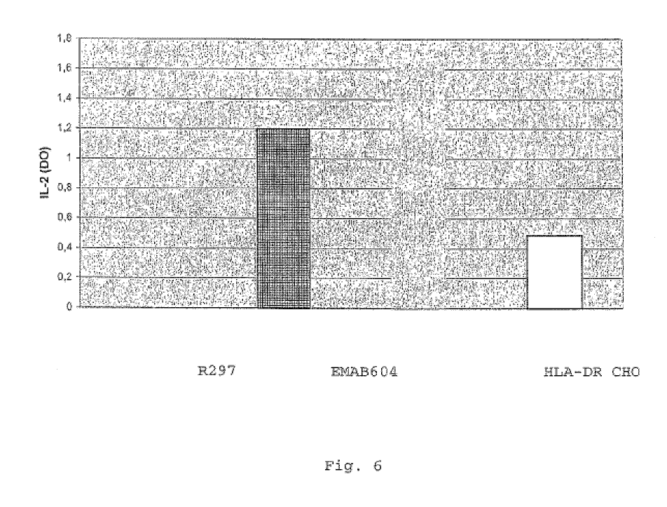

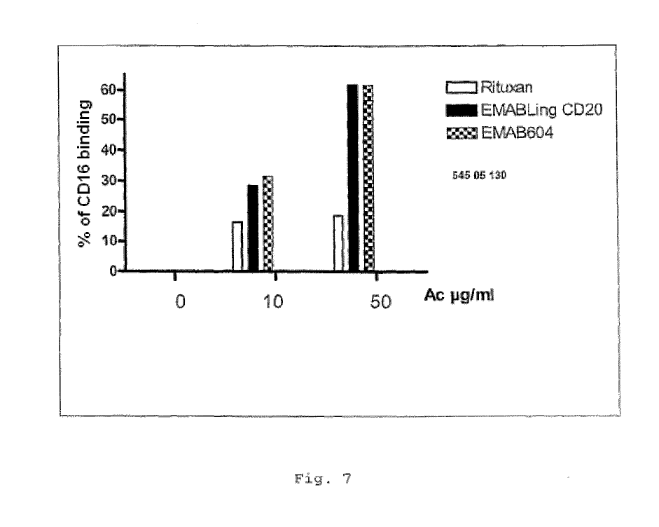

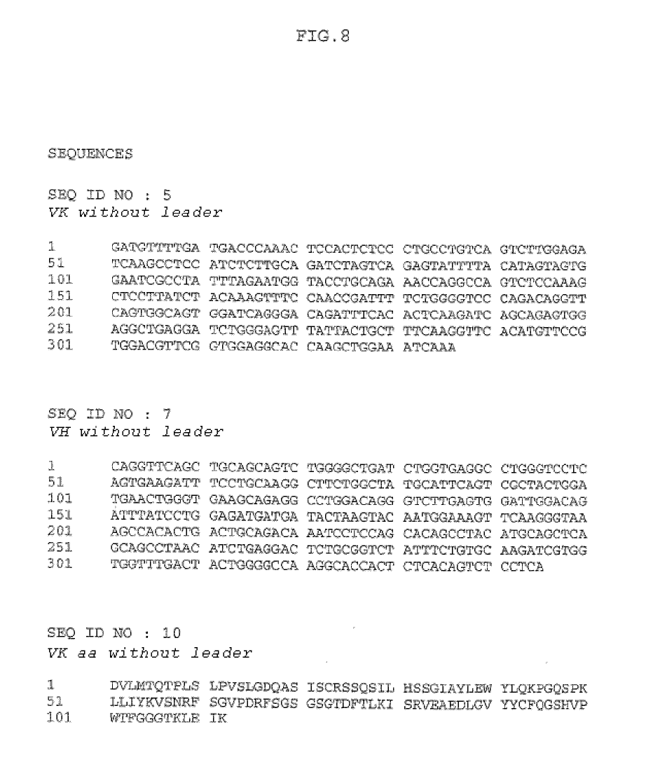

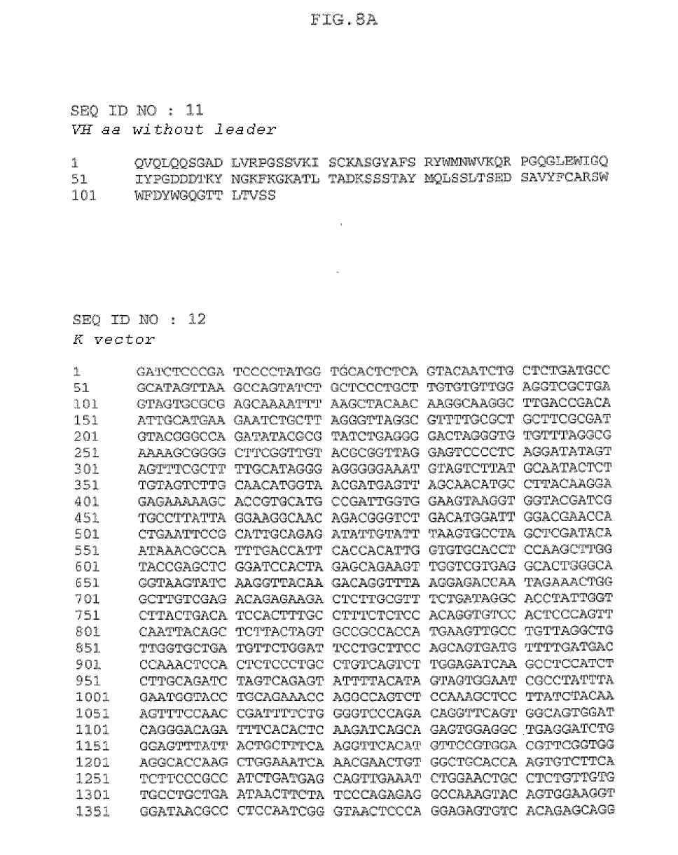

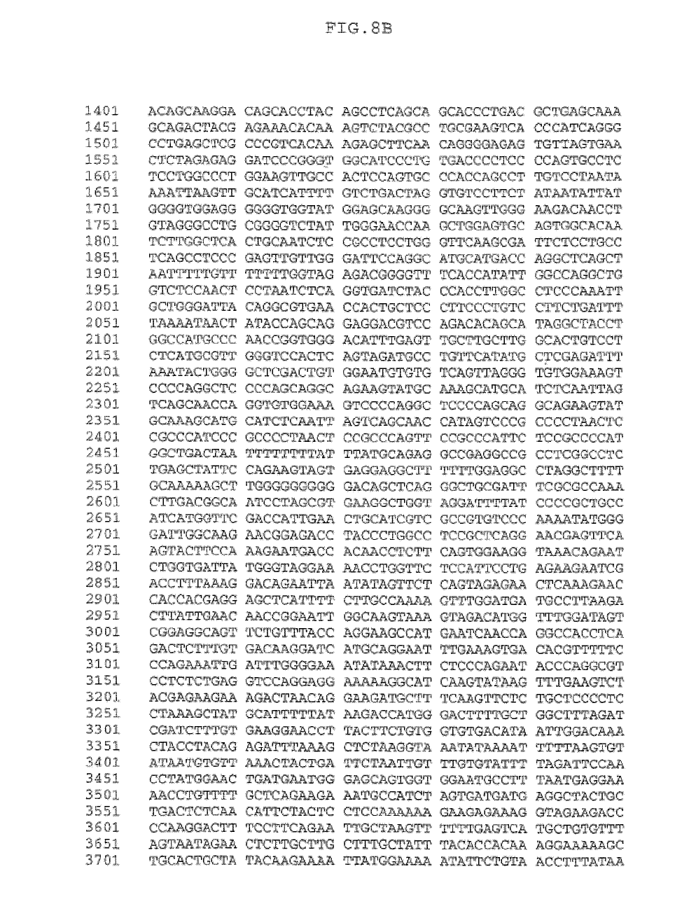

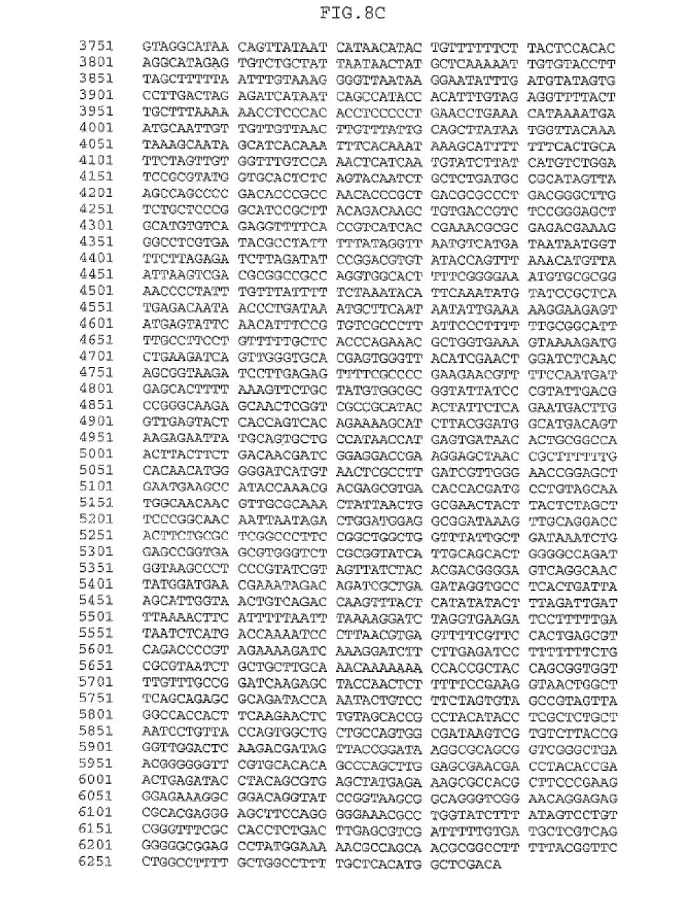

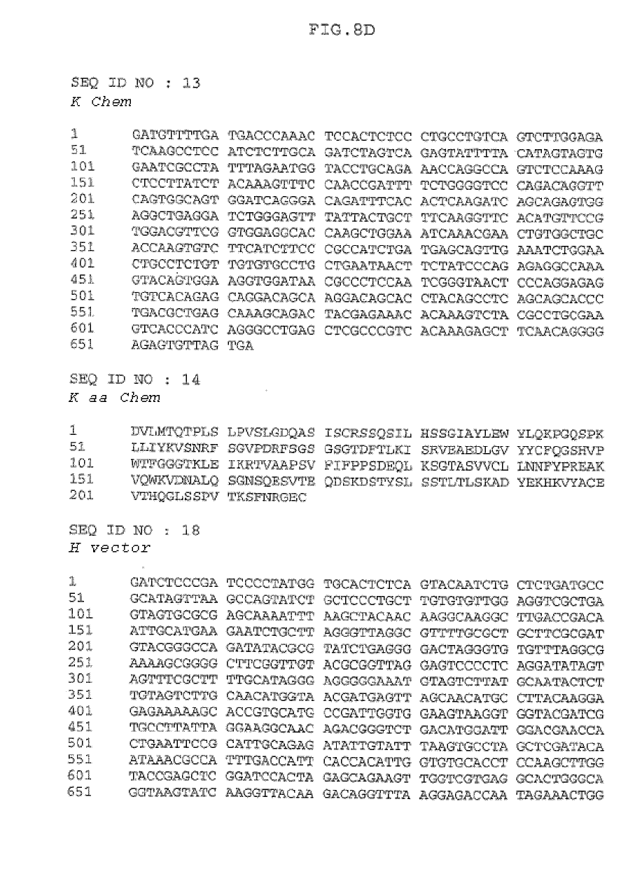

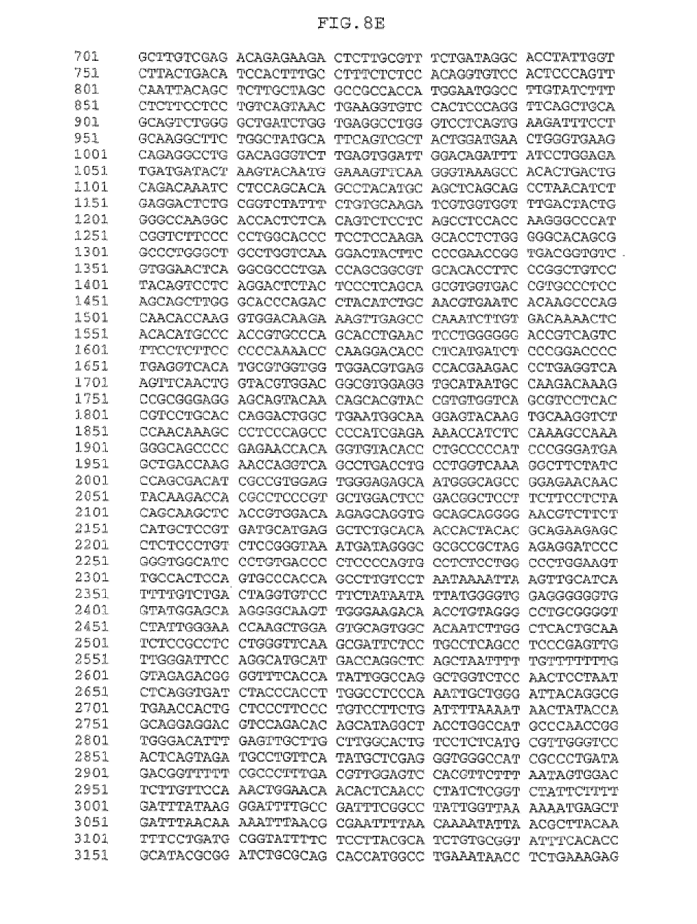

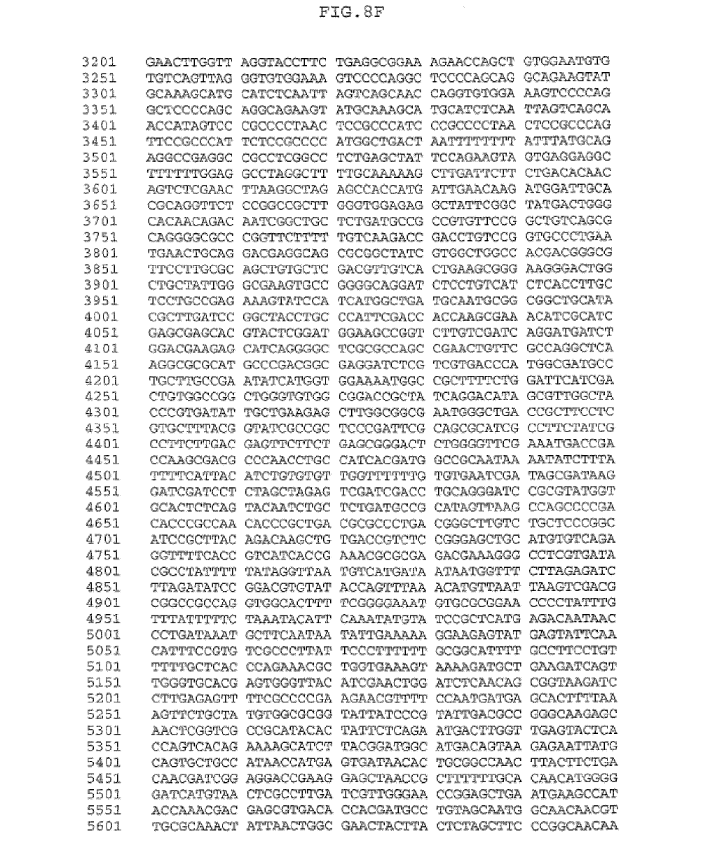

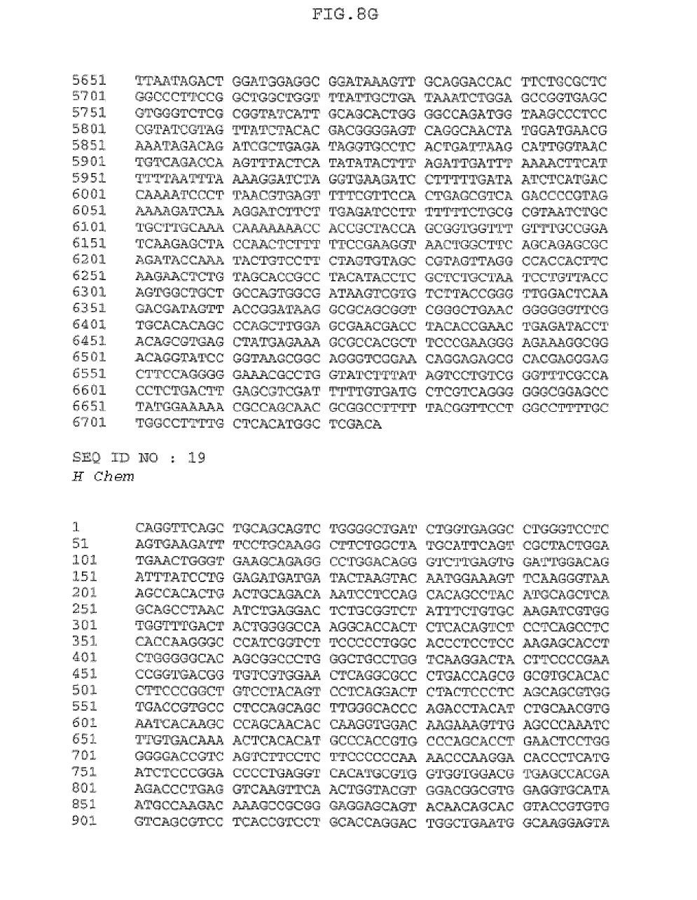

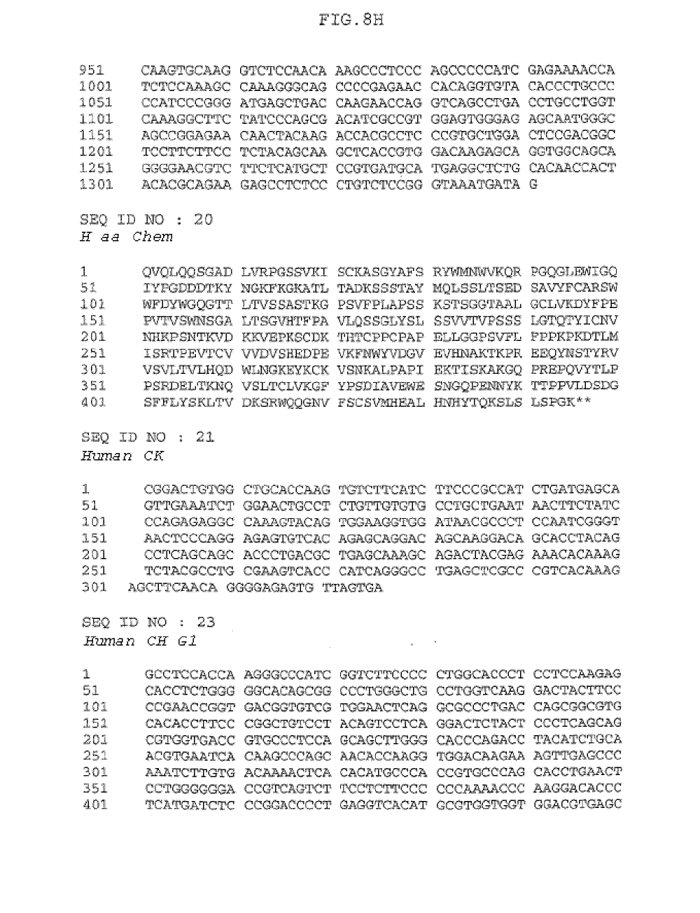

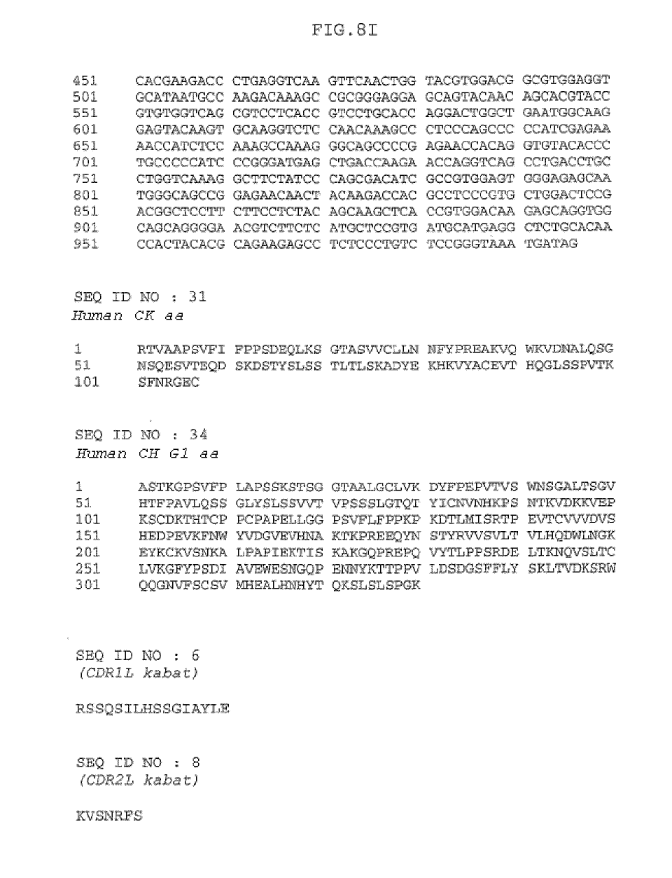

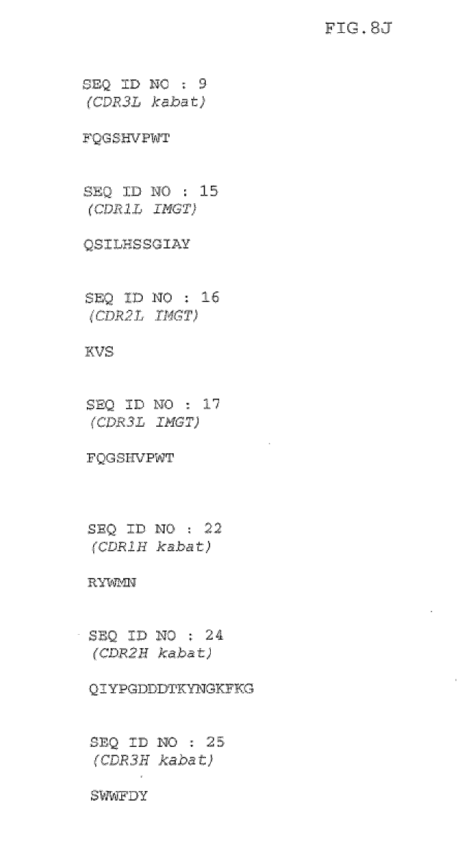

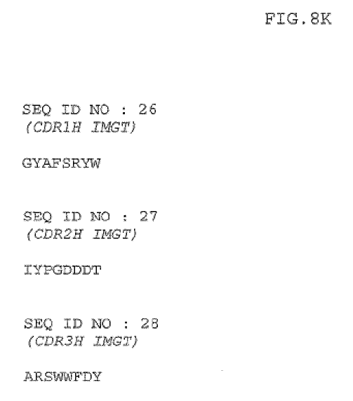

### Protocol for Implementation of the IL-2 Secretion Test (FIGS. 5 and 6)

This test uses a Jurkat line transfected with the human CD16 receptor (FcgammaRIIIa) as effector cell (Jurkat-CD16). The technique is based on measurement of the secretion of interleukin 2 (IL-2) by the Jurkat-CD16 line induced by the engagement of the CD16 with the tested antibodies bound to the target cells (melanoma lines HT-144 or GUY 17.2). To this end, 25 ng/ml of the antibodies tested are added to the target cells (1.5×10⁵ cells/ml) in the presence of the Jurkat-CD16 cells (5×10⁶ cells/ml) and 10 ng/ml of phorbol myristate acetate (PMA) for 18 hours at 37° C. in 5% CO₂. The culture plates are then centrifuged and the IL-2 released into the culture supernatant quantified by ELISA (Quantikine IL-2, R&D, Abingdon, UK).

The final result is expressed in absorbance units (OD).

| | HT 144 | R297 | EMAB604 | HLA-DR CHO |
|---|---|---|---|---|
| 25 ng/ml | | 0 | 1.199 | 0.49 |
| | GUY 17.2 | R297 | EMAB604 | HLA-DR CHO |
| 25 ng/ml | | 0.007 | 1.353 | 0.281 |

Table of results corresponding to FIGS. 5 and 6

### Example 7

### Ability of the EMAB604 and Anti-HLA-DR Antibodies Produced by the CHO Line on the Melanoma Line GUY 17.2 to Induce the Secretion of IL-2 by the Jurkat-CD16 Line (FIG. 5)

The antibody EMAB604 induces a secretion of IL-2 in the presence of the GUY 17.2 line greater than the same antibody produced in CHO.

The maximum lysis values (Emax) are 1.35 and 0.28 OD units for the EMAB604 and anti-HLA-DR CHO antibodies.

The anti-Rhesus D antibody R297 produced in YB2/0, serves as a negative control vis-à-vis the GUY 17.2 cells.

### Example 8

### Ability of the EMAB604 and Anti-HLA-DR Antibodies Produced by the Melanoma line HT-144 to Induce the Secretion of IL-2 by the Jurkat-CD16 Line (FIG. 6)

The antibody EMAB604 induces a secretion of IL-2 in the presence of the HT-144 line greater than the same antibody produced in CHO.

The maximum lysis values (Emax) are 1.2 and 0.49 OD units for the EMAB604 and anti-HLA-DR CHO antibodies.

The anti-Rhesus D antibody R297, produced in YB2/0, serves as a negative control vis-à-vis the HT-144 cells.

### Example 9

### Protocol for the CD16 Receptor Binding Test

The binding to the CD16 receptor is evaluated by a murine anti-CD16 antibody (3G8) competition test.

The effector cells (NK cells) are purified by the negative depletion kit (NK Cell Isolation Kit, Myltenyi, Paris, France), starting with peripheral blood from healthy donors. Then, the NK cells are incubated with variable concentrations (0, 10 and 50 μg/ml) of antibodies to be evaluated and the anti-CD16 antibody (3G8) coupled to a fluorochrome (3G8-PE) at a fixed concentration.

After washing, the binding of the 3G8-PE to the CD16 receptor of the NK cells is evaluated by flow cytometry. The results are expressed as a binding percentage, 100% binding corresponding to the total inhibition of the binding of the murine anti-CD16 antibody (3G8).

The antibody EMAB604 binds strongly to the CD16 of the NK cells in a way comparable to that of an anti-CD20 antibody produced by the EMABling platform (and described in the Patent Application WO 2006064121). This binding (62%) is approximately 3 times greater than that of Rituxan® (18.5%), an antibody produced in the CHO line, at a concentration of 50 μg/ml (FIG. 7).

1. Monoclonal antibody, monoclonal antibody fragment or monoclonal antibody derivative, directed against the human LDL (Low Density Lipoprotein) receptor, wherein the variable region of each of its light chains is encoded by the murine nucleic acid sequence SEQ ID NO: 5, the variable region of each of its heavy chains is encoded by the murine nucleic acid sequence SEQ ID NO: 7, or by nucleic acid sequences having sufficient homology with the sequences SEQ ID NO: 5 and SEQ ID NO: 7 for the nature and the binding affinity of said antibody to its antigen not to be modified, and the constant regions of its light chains and of its heavy chains are constant regions originating from a non-murine species.

2. Antibody according to claim 1, wherein the constant regions of each of its light chains and of each of its heavy chains are human constant regions.

3. Antibody according to claim 1, wherein the constant region of each of its light chains is of type κ.

4. Antibody according to claim 1, wherein the constant region of each of its heavy chains is of type γ.

5. Antibody according to claim 4, wherein the constant region of each of its heavy chains is of type γ1.

6. Antibody according to claim 5, wherein the constant region of each of its heavy chains is of type γ1 and is encoded by the nucleic acid sequence SEQ ID NO: 23 and in that the constant region of each of its light chains is encoded by the nucleic acid sequence SEQ ID NO: 21.

7. Antibody according to claim 1, wherein each of its light chains is encoded by the chimeric nucleic acid sequence SEQ ID NO: 13 and in that each of its heavy chains is encoded by the chimeric nucleic acid sequence SEQ ID NO: 19.

8. Antibody according to claim 7, wherein the peptide sequence deduced from the sequence SEQ ID NO: 13 is the sequence SEQ ID NO: 14 and in that the peptide sequence deduced from the sequence SEQ ID NO: 19 is the sequence SEQ ID NO: 20.

9. Antibody according to claim 1, wherein it is produced by a rat hybridoma cell line.

10. Antibody according to claim 9, wherein it is produced in the rat hybridoma YB2/0 (YB2/3HL.P2.G11.16Ag.20 cell, deposited at the American Type Culture Collection under number ATCC CRL-1662).

11. Antibody according to claim 10, wherein the antibody EMAB604 produced by the clone R604 deposited under registration number CNCM 1-3692 at the Collection Nationale de Cultures de Microorganismes (CNCM).

12. Expression vector of the light chain of an antibody as defined according to claim 1 of sequence SEQ ID NO: 12.

13. Expression vector of the heavy chain of an antibody as defined according to claim 1 of sequence SEQ ID NO: 18.

14. Stable cell line expressing an antibody according to claim 1.

15. Stable cell line according to claim 14, wherein the cell line in which the antibody is expressed is chosen from the group consisting of: SP2/0, YB2/0, IR983F, the human myeloma Namalwa, PERC6, the CHO lines, in particular CHO—K-1, CHO-Lec1O, CHO-Lec1, CHO-Lec13, CHO Pro-5, CHO dhfr-, Wil-2, Jurkat, Vero, MoIt-4, COS-7, 293-HEK, BHK, K6H6, NS0, SP2/0-Ag 14 and P3X63Ag8.653.

16. Stable cell line according to claim 14, having incorporated the two expression vectors according to claims 12 and 13.

17. Clone R604 deposited under registration number CNCM 1-3692 at the Collection Nationale de Cultures de Microorganismes (CNCM).

18. DNA fragment of sequence SEQ ID NO: 19 encoding for the heavy chain of an antibody according to claim 1.

19. DNA fragment of sequence SEQ ID NO: 13 encoding for the light chain of an antibody according to claim 1.

20. Use of an antibody according to claim 1, to activate in vitro the FcγRIII receptors of effector immune cells or cause the secretion of cytokines or chemokines by effector cells.

21. Antibody according to claim 1 for its use as a medicament.

22. Use of an antibody according to claim 1, for the production of a medicament intended for the treatment of cancer.

23. Use of an antibody according to claim 1, for the production of a medicament intended for the treatment of a melanoma.

24. Use of a monoclonal antibody directed against the human LDL receptor for the production of a medicament intended for the treatment of a melanoma.

25. Use of an antibody according to claim 23, in combination with one or more other antibody(ies) directed against one or more other antigen(s) expressed on the melanoma cells.

26. Use of an antibody according to claim 25, wherein said antigen expressed on the melanoma cells is chosen from the HLA-DR, CD20, CD22, CD23, CD25, CD30, CD33 and CD40.

27. Use according to claim 22, in vitro or ex vivo, of an antibody as defined according to any one of these claims, in combination with cells expressing FcγR, such as the NK (Natural Killer) cells, the NKT (Natural Killer T) cells, the Tγδ lymphocytes, the macrophages, the monocytes, any cell genetically modified to express CD 16 or the dendritic cells.

28. Pharmaceutical composition comprising at least one antibody according to claim 1 and at least one excipient and/or at least one pharmaceutically acceptable vehicle.

29. Composition according to claim 28, wherein it also comprises at least one antibody directed against another antigen present on the target cells of said antibody.

30. Composition according to claim 28, wherein it also comprises an anti-HLA-DR antibody.


SEQUENCE LISTING (34 sequences)

SEQ ID NO: 1 - DNA, 24 bp, Artificial Sequence
  Primer
actgccatcaatcttccacttgac

SEQ ID NO: 2 - DNA, 23 bp, Artificial Sequence
  Primer
gtgtagagtccagactgcaggag

SEQ ID NO: 3 - DNA, 28 bp, Artificial Sequence
  Primer
ttgttcaagaagcacacgactgaggcac

SEQ ID NO: 4 - DNA, 25 bp, Artificial Sequence
  Primer
cactgactcagggaagtagcccttg

SEQ ID NO: 5 - DNA, 336 bp, Mus musculus
gatgttttgatgacccaaactccactctccctgcctgtcagtcttggagatcaagcctccatctcttgcagatctagtcagagtattttacatagtagtggaatcgcctatttagaatggtacctgcagaaaccaggccagtctccaaagctccttatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 6 - protein, 16 aa, Mus musculus
RSSQSILHSSGIAYLE

SEQ ID NO: 7 - DNA, 345 bp, Mus musculus
caggttcagctgcagcagtctggggctgatctggtgaggcctgggtcctcagtgaagatttcctgcaaggcttctggctatgcattcagtcgctactggatgaactgggtgaagcagaggcctggacagggtcttgagtggattggacagatttatcctggagatgatgatactaagtacaatggaaagttcaagggtaaagccacactgactgcagacaaatcctccagcacagcctacatgcagctcagcagcctaacatctgaggactctgcggtctatttctgtgcaagatcgtggtggtttgactactggggccaaggcaccactctcacagtctcctca

SEQ ID NO: 8 - protein, 7 aa, Mus musculus
KVSNRFS

SEQ ID NO: 9 - protein, 9 aa, Mus musculus
FQGSHVPWT

SEQ ID NO: 10 - protein, 112 aa, Mus musculus
DVLMTQTPLSLPVSLGDQASISCRSSQSILHSSGIAYLEWYLQKPGQSPKLLIYKVSNRFSGVPDRFSGSGSGTDFTLKISRVEAEDLGVYYCFQGSHVPWTFGGGTKLEIK

SEQ ID NO: 11 - protein, 115 aa, Mus musculus
QVQLQQSGADLVRPGSSVKISCKASGYAFSRYWMNWVKQRPGQGLEWIGQIYPGDDDTKYNGKFKGKATLTADKSSSTAYMQLSSLTSEDSAVYFCARSWWFDYWGQGTTLTVSS

SEQ ID NO: 12 - DNA, 6288 bp, Mus musculus
gatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagcttggtaccgagctcggatccactagagcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttactagtgccgccaccatgaagttgcctgttaggctgttggtgctgatgttctggattcctgcttccagcagtgatgttttgatgacccaaactccactctccctgcctgtcagtcttggagatcaagcctccatctcttgcagatctagtcagagtattttacatagtagtggaatcgcctatttagaatggtacctgcagaaaccaggccagtctccaaagctccttatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaacgaactgtggctgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgaactctagagaggatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcatatgctcgagatttaaatactggggctcgactgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggggggggggacagctcagggctgcgatttcgcgccaaacttgacggcaatcctagcgtgaaggctggtaggattttatccccgctgccatcatggttcgaccattgaactgcatcgtcgccgtgtcccaaaatatggggattggcaagaacggagacctaccctggcctccgctcaggaacgagttcaagtacttccaaagaatgaccacaacctcttcagtggaaggtaaacagaatctggtgattatgggtaggaaaacctggttctccattcctgagaagaatcgacctttaaaggacagaattaatatagttctcagtagagaactcaaagaaccaccacgaggagctcattttcttgccaaaagtttggatgatgccttaagacttattgaacaaccggaattggcaagtaaagtagacatggtttggatagtcggaggcagttctgtttaccaggaagccatgaatcaaccaggccacctcagactctttgtgacaaggatcatgcaggaatttgaaagtgacacgtttttcccagaaattgatttggggaaatataaacttctcccagaatacccaggcgtcctctctgaggtccaggaggaaaaaggcatcaagtataagtttgaagtctacgagaagaaagactaacaggaagatgctttcaagttctctgctcccctcctaaagctatgcatttttataagaccatgggacttttgctggctttagatcgatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagagatcttagatatccggacgtgtataccagtttaaacatgttaattaagtcgacgcggccgccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgaca

SEQ ID NO: 13 - DNA, 663 bp, Artificial Sequence
  Synthetic Construct
gatgttttgatgacccaaactccactctccctgcctgtcagtcttggagatcaagcctccatctcttgcagatctagtcagagtattttacatagtagtggaatcgcctatttagaatggtacctgcagaaaccaggccagtctccaaagctccttatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaacgaactgtggctgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 14 - protein, 219 aa, Artificial Sequence
  Synthetic Construct
DVLMTQTPLSLPVSLGDQASISCRSSQSILHSSGIAYLEWYLQKPGQSPKLLIYKVSNRFSGVPDRFSGSGSGTDFTLKISRVEAEDLGVYYCFQGSHVPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 11 aa, Mus musculus
QSILHSSGIAY

SEQ ID NO: 16 - protein, 3 aa, Mus musculus
KVS

SEQ ID NO: 17 - protein, 9 aa, Mus musculus
FQGSHVPWT

SEQ ID NO: 18 - DNA, 6726 bp, Mus musculus
gatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagcttggtaccgagctcggatccactagagcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttgctagcgccgccaccatggaatggccttgtatctttctcttcctcctgtcagtaactgaaggtgtccactcccaggttcagctgcagcagtctggggctgatctggtgaggcctgggtcctcagtgaagatttcctgcaaggcttctggctatgcattcagtcgctactggatgaactgggtgaagcagaggcctggacagggtcttgagtggattggacagatttatcctggagatgatgatactaagtacaatggaaagttcaagggtaaagccacactgactgcagacaaatcctccagcacagcctacatgcagctcagcagcctaacatctgaggactctgcggtctatttctgtgcaagatcgtggtggtttgactactggggccaaggcaccactctcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatagggcgcgccgctagagaggatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttcatatgctcgagggtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatcgatcctctagctagagtcgatcgacctgcagggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagagatcttagatatccggacgtgtataccagtttaaacatgttaattaagtcgacgcggccgccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgaca

SEQ ID NO: 19 - DNA, 1341 bp, Artificial Sequence
  Synthetic Construct
caggttcagctgcagcagtctggggctgatctggtgaggcctgggtcctcagtgaagatttcctgcaaggcttctggctatgcattcagtcgctactggatgaactgggtgaagcagaggcctggacagggtcttgagtggattggacagatttatcctggagatgatgatactaagtacaatggaaagttcaagggtaaagccacactgactgcagacaaatcctccagcacagcctacatgcagctcagcagcctaacatctgaggactctgcggtctatttctgtgcaagatcgtggtggtttgactactggggccaaggcaccactctcacagtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatag

SEQ ID NO: 20 - protein, 445 aa, Artificial Sequence
  Synthetic Construct
QVQLQQSGADLVRPGSSVKISCKASGYAFSRYWMNWVKQRPGQGLEWIGQIYPGDDDTKYNGKFKGKATLTADKSSSTAYMQLSSLTSEDSAVYFCARSWWFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 327 bp, Homo sapiens
cggactgtggctgcaccaagtgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtga

SEQ ID NO: 22 - protein, 5 aa, Mus musculus
RYWMN

SEQ ID NO: 23 - DNA, 996 bp, Homo sapiens
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgatag

SEQ ID NO: 24 - protein, 17 aa, Mus musculus
QIYPGDDDTKYNGKFKG

SEQ ID NO: 25 - protein, 6 aa, Mus musculus
SWWFDY

SEQ ID NO: 26 - protein, 8 aa, Mus musculus
GYAFSRYW

SEQ ID NO: 27 - protein, 8 aa, Mus musculus
IYPGDDDT

SEQ ID NO: 28 - protein, 8 aa, Mus musculus
ARSWWFDY

SEQ ID NO: 29 - DNA, 40 bp, Artificial Sequence
  Primer
atcgaactagtgccgccaccatgaagttgcctgttaggct

SEQ ID NO: 30 - DNA, 52 bp, Artificial Sequence
  Primer
gatgaagacacttggtgcagccacagttcgtttgatttccagcttggtgcct

SEQ ID NO: 31 - protein, 107 aa, Homo sapiens [sequence omitted]

SEQ ID NO: 32 - DNA, 40 bp, Artificial Sequence
  Primer
atcgagctagcgccgccaccatggaatggccttgtatctt

SEQ ID NO: 33 - DNA, 41 bp, Artificial Sequence
  Primer
accgatgggcccttggtggaggctgaggagactgtgagagt

SEQ ID NO: 34 - protein, 330 aa, Homo sapiens [sequence omitted]